(19) United States
(12) Patent Application Publication
(10) Pub. No.: US 2009/0203685 A1
(43) Pub. Date: Aug. 13, 2009

(54) ANTI-PROLIFERATIVE COMPOUNDS FROM A 3-ARYL-COUMARINE OR 3-ARYL-QUINOLIN-2-ONE AND USES THEREOF

(75) Inventors: Matthieu Schapira, Lyon (FR); Cyrille Lamigeon, Saint Priest (FR); Mathieu Gutmann, Lyon (FR); Audrey Barthelaix, Lyon (FR); Nicolas Hugo, Lyon (FR); Pierre Colas, St Didier au Mont d'Or (FR)

Correspondence Address: ROTHWELL, FIGG, ERNST & MANBECK, P.C., 1425 K STREET, N.W., SUITE 800, WASHINGTON, DC, 20005, US

(73) Assignee: EOS (ETHICAL ONCOLOGY SCIENCE) S.P.A., Milano (IT)

(21) Appl. No.: 12/301,711

(22) PCT Filed: May 22, 2007
(86) PCT No.: PCT/IB2007/002179

§ 371 Date: Feb. 6, 2009

(30) Foreign Application Priority Data
May 22, 2006 (EP) 06290821.5

(51) Int. Cl. A61K 31/5377; A61K 31/4709; A61K 31/366; C07D 413/12; C07D 311/06; C07D 215/227; C07D 407/12; A61K 31/4704; C07D 405/12

(52) U.S. Cl. 514/233.5; 514/312; 514/456; 544/151; 546/157; 549/290

(57) ABSTRACT

The present invention is directed to molecules deriving from a 3-aryl-coumarine or 3-aryl-quinolin-2-one and having potent anti-proliferative and/or cytotoxic activity, especially against tumoral cells. The present invention also concerns the uses of these molecules in therapeutic application, for the treatment of different cancers. The invention also discloses the use of said compound for the manufacture of a medicament for treating cancer. The invention also concerns a process for inhibiting cell proliferation comprising contacting said cells with a compound of the invention.

Cancer is one of the most widespread diseases around the world. Although the treatments proposed to patients are making progress, medicaments remain in many cases poorly effective, the treatments are usually difficult to bear due to dramatic side effects, and often do not avoid the chirurgical ablation of the tissue or organ affected by tumor cells. Patient death is still a frequent occurrence.

Indeed, despite recent advances in the diagnosis and treatment of cancer, tumor cell progression and metastasis still constitute a major cause of morbidity and mortality in cancer patients. Understanding the molecular and cellular mechanisms that contribute to tumor formation, progression and metastasis is a major challenge in cancer research, as is the need to inhibit the proliferation of tumoral cells.

Most of the anticancer drugs available to date kill tumor cells by activating an endogenous biochemical pathway for cell suicide, known as programmed cell death or apoptosis. Apoptosis is a genetically-programmed cell death. Apoptosis is also important in controlling cell number and proliferation as part of normal development. Although some chemotherapeutic agents do not induce apoptosis directly, this programmed cell death is often the final common pathway for cells that have been damaged in various ways by cytotoxic drugs.

However, many malignant cells develop defects in the regulation of genes that control the apoptotic pathway, thus rendering such cells resistant to chemotherapy.

A number of mechanisms have been proposed for tumor insensitivity to the effects of conventional chemotherapy agents, including multidrug resistance gene expression, membrane pump extrusion of chemotherapeutic agents, tumor hypoxia, decreased tumor angiogenesis, and unfavorable cell cycle kinetics. Resistance to apoptosis is another mechanism via which tumors may both avoid programmed senescence and death as well as chemotherapy effects. It has been reported that the development of apoptosis resistance can lead to an impaired response to conventional chemotherapeutic agents.

Although there are multiple pathways via which a cell can enter apoptosis, it appears that the majority of chemotherapy drugs induce apoptosis via effects on the mitochondrion (cytochrome c activation of cellular caspases) rather than cell surface death receptors such as FasL. The BCL-2 family of proteins are the major regulators of mitochondrial apoptotic homeostasis. The interplay between phosphorylating events, mediated largely by the phosphatidyl inositol 3 (PI3) kinase system, as well as the balance achieved between pro-apoptotic and anti-apoptotic BCL-2 proteins determines whether or not the cell enters or avoids the process of programmed cell death.

The BCL-2 family of proto-oncogenes is a critical regulator of apoptosis, whose expression frequently becomes altered in human cancers, including lymphomas. Bcl-2 was the first member of this family to be identified, by virtue of its involvement in chromosomal translocation commonly found in B-cell non-Hodgkin's lymphoma (NHL).

The BCL-2 family is a large family of proteins subdivided into two subfamilies, which share some regions of homology known as BCL-2 Homology (BH) regions, a first class being anti-apoptotic, and a second class being pro-apoptotic. Their main mechanism of action is thought to be the regulation of mitochondrial membrane permeability. Pro-apoptotic members of the BCL-2 superfamily increase mitochondrial membrane permeability, whereas anti-apoptotic members of the family act to oppose this increase.

Pro-apoptotic members of the BCL-2 family include Bax, Bak, Bad, Bcl-Xs, Bid, Bik, Bim, and Hrk, and anti-apoptotic members include Bcl-2, Bcl-xL, Bcl-W, Bfl-1, and Mcl-1.

Anti-apoptotic BCL-2 members act as repressors of apoptosis by blocking the release of cytochrome-c, whereas pro-apoptotic members act as promoters. There are several possible mechanisms by which members of the BCL-2 family could regulate mitochondrial membrane permeability.

The anti-apoptotic members of the BCL-2 family are overexpressed in many cancers even without the presence of chromosomal translocations. Increased expression of anti-apoptotic BCL-2 members causes resistance to chemotherapeutic drugs and radiation therapy, while decreasing anti-apoptotic BCL-2 members expression may promote apoptotic responses to anticancer drugs. In addition, overexpression of Bcl-2, an anti-apoptotic member, may result in accumulation of cells in the G₀ phase of cell cycle division and contribute to chemoresistance. With regard to Bcl-xL, an anti-apoptotic member of the BCL-2 family, it has been previously demonstrated that:
Bcl-xL is a major anti-apoptotic protein
Bcl-xL (and other BCL-2-like proteins) is found over-expressed in many cancers
Basal expression of Bcl-xL correlates strongly with chemoresistance to various classes of anti-tumor molecules.

Therefore, small molecule inhibitors of anti-apoptotic activity of Bcl-xL are expected to exert an anti-tumor effect on a wide range of cancers, either alone or in combination with existing cytotoxic agents.

The BCL-2 family of proteins, and especially Bcl-xL are thus attractive targets for drug design. As pivotal regulators of apoptotic cell death, the logic of manipulating BCL-2 functions for anti-tumor effects is one of the molecular targets proposed for cancer therapeutics. Moreover, elevated levels of anti-apoptotic proteins have been demonstrated in virtually every type of human cancer.

Initial efforts have been centered on disrupting protein-protein interactions within the BCL-2 homology (BH) family. Substantial progress in this task has been made using molecular modeling and drug leads.

Different molecules targeting proteins of the BCL-2 family are presently in preclinical or clinical trials, inter alia:
ABT-737 (Oltersdorf et al, 2005): Molecular targets: Bcl-xL, Bcl-2, Bcl-w. ABT-737 induces apoptosis of small cell lung carcinoma (SCLC) and lymphoid cells and induces SCLC tumor regression in mouse models.
IPI-983L (http://www.ipi.com/science_bio.html): Molecular targets: Bcl-xL, Bcl-2.
Gossypol (Mohammad R M et al, 1995): Molecular targets: BCL-2 like proteins. This molecule has been shown to inhibit growth of breast and prostate tumor cell lines in vitro.
GX15-070 (Contractor R. et al, and Galan P P et al): Molecular targets: BCL-2-like proteins (Kd for Bcl-w and MCL-1˜0.5 mM). This molecule has been shown to inhibit growth of melanoma, breast, cervical, prostate, colon tumor cells. It has however a low MTD (maximal tolerated dose of approx 16 mg/kg).

The present inventors have now obtained a novel family of potent anti-cancer molecules targeting Bcl-xL. The family of molecules is derived from a 3-aryl-coumarine or 3-aryl-quinolin-2-one. The present invention is directed to these compounds and to their uses in therapy, especially for treating any type of cancer.

In the context of the present invention, the following terms are defined according to the following definitions:

Apoptosis is defined as programmed cell death, and involves the systematic disassembly of a cell. In contrast to necrosis, which is a form of cell death that results from acute cellular injury, apoptosis is carried out in an ordered process that generally confers advantages during an organism's life cycle.

The main function of apoptosis is to dispose of a cell without causing damage or stress to neighbouring cells.

Apoptosis involves the systematic dissassembly of the cell into apoptotic bodies, which are taken up by phagocytes. This avoids the inflammatory response associated with necrosis. Apoptosis can be detected using the assay described in example 7 of the experimental part.

Anti-apoptotic: Something that prevents, decreases or delays apoptosis.

Pro-apoptotic: Something that activates or accelerates apoptosis.

Proliferation: multiplication or increase in number. In biology, cell proliferation occurs by cell division.

Anti-proliferative: something that prevents, decreases or delays proliferation of cells; therefore after a treatment with an anti-proliferative compound, the number of cells remains static, or increases less rapidly than without treatment, or decreases. An anti-proliferative activity may be due to a cytostatic effect, to a cytotoxic effect, or to a combination of both effects. The anti-proliferative activity of a compound can be tested and measured according to the protocol detailed in the experimental part of the description (see example 2C). A molecule is said to have anti-proliferative activity according to the present invention if the number of cells is significantly diminished (from a statistical point of view) in a sample treated during 72 hours with said molecule with respect to the number of cells in a sample treated during 72 hours with a control, or untreated.

Cytotoxic: something producing a lethal effect on cells. A compound is considered as cytotoxic, if after treatment for 72 hours with said compound the number of cells is significantly lower (from a statistical point of view) than the number of cells initially seeded. Example 2A of the specification details a potential protocol for determining the cytotoxic activity of a compound. According to the invention, a compound having cytotoxic properties is an example of an anti-proliferative compound.

Cytotoxicity may lead to the death of the cell inter alia by necrosis or apoptosis.

Cytostatic: something inhibiting or suppressing cellular division. According to the invention, a compound having cytostatic properties is an example of an anti-proliferative compound.

Cancer: Any of various malignant neoplasms characterized by the proliferation of anaplastic cells that tend to invade surrounding tissue and metastasize to new body sites.

Anticancer or anticancerous: Effective in treating cancer.

Tumor: An abnormal growth of tissue resulting from uncontrolled, progressive multiplication of cells and serving no physiological function; a neoplasm.

Antitumor or antitumoral: Counteracting or preventing the formation of malignant tumors; anticancer.

Alkyl represents straight and branched carbon chains that contain from one to twelve carbon atoms, preferably one to six carbon atoms; most preferably 1 to 5. A lower alkyl contains one to four carbon atoms, it is thus a C₁, C₂, C₃ or C₄ carbon chain.

Cycloalkyl represents saturated carbocyclic rings branched or unbranched of from 3 to 20 carbon atoms, preferably 3 to 7 carbon atoms; most preferably 5 or 6; said carbocyclic ring being optionally substituted with one or more of halo, alkyl, hydroxy, alkoxy, phenoxy, CF3, amino, alkylamino, dialkylamino, —COOR, —NO₂ (wherein R is H or alkyl), C≡N, OH, O—CH₃, CO—NH₂, CO—OH, CO—OCH₃ or O—CO—CH₃; said carbocyclic ring being preferably mono- or di-substituted. A cycloalkyl possesses preferably less than 200 atoms or less than 100 atoms, even preferably less than 60 atoms, or less than 40 atoms.

Heterocycloalkyl represents a saturated, branched or unbranched carbocylic ring containing from 3 to 15 carbon atoms, preferably from 4 to 6 carbon atoms, which carbocyclic ring is interrupted by 1 to 3 hetero groups selected from —O—, —S— or —NR— (wherein R can be, for example, —C(O)N(H)₂, —CH₂C(O)N(H)₂, —CHO, —C(O)—O—H, alkyl, aryl, aralkyl, cycloalkyl, heterocycloalkyl or heteroaryl). Preferably, a heterocycloalkyl is a carbocyclic ring interrupted by 1 or 2 hetero groups. A heterocycloalkyl possesses preferably less than 200 atoms or less than 100 atoms, even preferably less than 60 atoms, or less than 40 atoms.

Preferred heterocycloalkyls are furan, thiophene, pyrrole, thiazole, pyrrazole and oxazole.

Aryl (including the aryl portion of aryloxy and aralkyl) represents a carbocyclic group containing from 6 to 15 carbon atoms and having at least one aromatic ring, said carbocyclic group being optionally substituted with one or more of halo, alkyl, heterocycloalkyl (e.g. N-alkyl-piperazine, morpholine, . . . ), hydroxy, alkoxy, phenoxy, CF3, amino, alkylamino, dialkylamino, —COOR (wherein R is H, alkyl, aryl, heteroaryl or cycloalkyl), NO₂, C≡N, OH, O—CH₃, CO—NH₂, CO—OH, CO—OCH₃ or O—CO—CH₃; said carbocylcic group is preferably mono- or di-substituted. An aryl possesses preferably less than 200 atoms or less than 100 atoms, even preferably less than 60 atoms, or less than 40 atoms.

Heteroaryl represents a cyclic group, optionally substituted, having at least one heteroatom selected from O, S or N, said heteroatom interrupting a carbocyclic ring structure and having a sufficient number of delocalized pi electrons to provide aromatic character, with the aromatic heterocyclic groups preferably containing from 2 to 14 carbon atoms. The aromatic heterocyclic group is optionally substituted with one or more of the substituents mentioned in the definition of aryl. Said carbocyclic ring is preferably interrupted by 1 or 2 heteroatoms. A heteroaryl possesses preferably less than 200 atoms or less than 100 atoms, even preferably less than 60 atoms, or less than 40 atoms.

Aralkyl (shortened form of arylalkyl) represents groups made up of both aryl and alkyl groups.

### LEGEND OF FIGURES

FIGS. 1: (1A and 1B) Inhibition of cell proliferation in vitro.

This graph represents the percentage of growth of fourteen different populations of cells, in presence of increasing concentration of a compound of the invention: compound (01).

FIG. 2: Inhibition of tumor growth in vivo.

This graph represents the growth of tumor size (by reference to the initial size of the tumor) grafted to mice receiving different treatments during 15 days.

Rhombus: evolution of the growth of the tumor measured in mice receiving the vehicle (without active ingredient) intra-peritoneally (2QD)

Triangle: evolution of the growth of the tumor measured in mice receiving the compound (01) intra-peritoneally at a dosage of 18 mg/kg (2QD).

Circle: evolution of the growth of the tumor measured in mice receiving taxol intra-peritoneally at 8 mg/kg (Q3D×5; d1; d4; d7; d10; d13).

Cross: evolution of the growth of the tumor measured in mice receiving a combination of compound (01) at a dosage of 18 mg/kg 2QD and taxol at a dosage of 8 mg/kg Q3D×5 intra-peritoneally.

FIG. 3: Inhibition of the interaction between Bcl-xL and BF8, a peptide aptamer that targets the hydrophobic groove of BCL-2 family members.

This picture is a Western blot analysis showing the presence, or absence of the protein fusion 6×His-Bcl-xL, captured by GST-BF8, in presence or in absence of a peptide of the invention, specifically compounds (01) and (02).

C−: control peptide aptamer (does not interact with Bcl-xL).
C+: 6×His-Bcl-xL captured by GST-BF8, in presence of 5% DMSO.
(02): 6×His-Bcl-xL captured by GST-BF8, in presence of 50 μM of compound 02 (in 5% DMSO).
(01): 6×His-Bcl-xL captured by GST-BF8, in presence of 50 μM of compound 01 (in 5% DMSO).

FIG. 4: This figure depicts the time course of cellular effects induced by 0.5 μM of compound 01 in U937 cells.

X-axis: time of exposure to compound (01), in hours.

Y-axis: % of positive cells

By using virtual screening in silico, the present inventors have identified a molecule capable of interacting in silico, with the Bcl-xL protein, whose three-dimensional structure is known (Sattler et al). This molecule has then been tested in vitro and in vivo and has proven to be an efficient anti-proliferative agent.

Specifically, the compound designated 01 inhibits the proliferation of a large panel of human cancer cells in vitro. Compound 01 also inhibits the growth of a non-small cell lung carcinoma in an in vivo xenograft model in the mouse.

Lead optimization has allowed the identification of a family of molecules, structurally related to compound 01, having comparable properties, useful in the treatment of cancer.

The compound 01 is (2E)-3-(2-furyl)-N-({[3-methoxy-4-(2-oxo-2H-chromen-3-yl)phenyl]amino}carbonothioyl)acrylamide.

A number of derivatives, keeping the core structure, have now been tested by the inventors, in order to enhance the advantageous anti-proliferative properties of the compound 01 and improve its suitability as a drug. All these molecules essentially possess the following structural core:

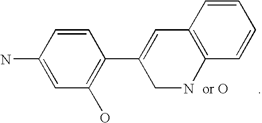

According to a first aspect, the present invention is thus directed to compounds having one of the general formula:

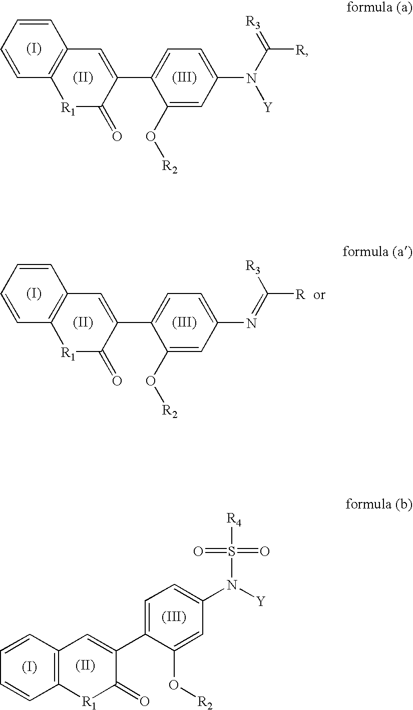

wherein the cycles (I), (II) and (III) may be optionally substituted on 1, 2, 3 or 4 of the available carbons with a group chosen amongst: Br, Cl, F, I, OH, O—CH₃, COOH, and (CH₂)ₘ—R₉, wherein R₉ represents H, CH₃, OH, O—CH₃, COOH, NH₃, CO—NH₂, NH—CO-phenyl, an alkyl, an heteroaryl or an heterocycloalkyl and m=1, 2, 3 or 4.
wherein Y represents H, CH₃ or CO—CH₃;
wherein R₁ represents O, NR₁₆ with R₁₆ being H or an alkyl having from 1 to 4 carbon atoms, or N—(CH₂)ₚ—R₇, with R₇ representing OH, CCOR₁₆, CO—NHR₁₆, NH₂R₁₆, O—CH₂R₁₆, CN, O—CO—CH₂R₁₆, CO-heterocycloalkyl, a cycloalkyl, a heterocycloalkyl, an aryl or a heteroaryl, possibly substituted with one or more of the followings radicals: an alkyl, an aryl, a heteroaryl, a cycloalkyl, a heterocycloalkyl, F, Cl, NH₂, C≡N, OH, CO—NH₂, O—CH₃, CO—OH, CO—OCH₃ or O—CO—CH₃, and p=0, 1, 2, 3, 4, 5 or 6 and with R₁₆ being H or an alkyl with 1, 2, 3 or 4 carbon atoms;
wherein R₂ represents H or an alkyl with less than 4 carbon atoms,
wherein R₃ represents S, O or NH in formula (a), or S—(CH₂)_{q}—R₁₀ or NH—(CH₂)_{q}—R₁₀, in formula (a′), wherein R₁₀ has the same meaning as R₉ and wherein q=0, 1, 2, 3 or 4;
wherein R₄ represents an alkyl, aryl, arylalkyl, heteroaryl, heteroarylalkyl, cycloalkyl, heterocycloalkyl, or CH═CH—R₁₁, wherein R₁₁ has the same meaning as R₄, and the molecular weight of R₄ is less than 500 Da,
and wherein,
   if R₃ represents O, then R is N*—R₅, or O—R₅, wherein R₅ has the same meaning as R₄ and N* represents NH or N(CH₃);
   if R₃ represents NH, S—(CH₂)_{q}—R₁₀ or NH—(CH₂)_{q}—R₁₀, then R is R₆, or N*—CO—R₆, wherein R₆ has the same meaning as R₄ and N* represents NH or N(CH₃); and
   if R₃ represents S, then
      R is N*—(CH₂)ₙ—R₁₂, wherein R₁₂ has the same meaning as R₇, n=0, 1, 2, 3 or 4 and N* represents NH or N(CH₃), or
      R is N*—CO—R₈, with -R₈ representing a linear or branched alkyl, with the exception of tert-butyl, or -R₈ represents —(CH₂)ₙ—C₄H₃O, with s=1, 2, 3 or 4, or R₈ represents a phenyl radical mono-substituted in the para position, with an alkyl, —F, —Cl, —NH₂, —C≡N, —OH, —CO—NH₂, —O—CH₃, —CO—OH, —CO—OCH₃ or —O—CO—CH₃ and N* represents NH or N(CH₃), or
      R is N*—CO—R₁₇, with -R₁₇ representing an alkyl, aryl, arylalkyl, heteroaryl, heteroarylalkyl, cycloalkyl, heterocycloalkyl, or CH═CH—R₁₈, wherein R₁₈ has the same meaning as R₁₇, and the molecular weight of R₁₇ is less than 500 Da, N* represents NH or N(CH₃) and R₁ is not O,
         as well as base and acid addition salts of said compounds, the geometric isomers, enantiomers and diastereoisomers thereof.

Each substitutent is chosen independently of the others.

According to an embodiment of the invention, the molecule has the general formula (a).

According to another embodiment, a molecule of the invention has the general formula (a′).

According to still another embodiment, a molecule of the invention has the formula (b).

The overall molecular mass of the compound is preferably less than 1000 Da. Preferably, the molecular weight is less than 800 Da, preferably less than 700, 650 or 600 Da. Indeed, the compound is preferably capable of passing (passively) the cell membrane. Therefore, the molecular weight of the compound is if possible minimized.

Preferably, a compound according to this first aspect of the invention has anti-proliferative properties, it may thus be useful as an antitumoral agent. The compounds of the invention have preferably an anti-proliferative effect mainly targeted on tumour cells, cancerous cells or immortalized cells, i.e. the anti-proliferative (or cytotoxic effect) is at least 10% greater on a population of tumour or cancerous cells than on a population of normal cells (not immortalized). For example, after incubating a population of cells with a compound according to this aspect of the invention, the decrease in the proliferation rate of the cells is 10% greater on a population of tumour or cancerous cells than on a population of normal cells. The difference in the effect on a population of tumour or cancerous cells with a population of normal cells is preferably at least 20%, at least 30% or at least 50% or even more.

Anti-proliferative activity can be tested and measured according to the protocol detailed in the experimental part of the description (see example 2C).

More preferably, said compounds have cytotoxic activity or pro-apoptotic activity. Preferably, this activity is preferentially targeted on tumour cells. Similarly, this means that the cytotoxic or pro-apoptotic effect is at least 10% greater on a population of tumour or cancerous cells than on a population of normal cells.

Pro-apoptotic activity can be tested and measured according to the protocol detailed in the experimental part of the description (example 7).

Preferably, a compound as defined according to this aspect of the invention is for use in therapy, in a prophylactic or curative treatment.

The compounds as defined are indeed useful for eliminating cancerous cells; they are thus very useful in the treatment of cancers, leukaemia or any type of tumours.

The compounds according to this first aspect of the invention are preferably to be administered to a patient, which is an animal, preferably a mammal and most preferably a human being. Therefore, substituents likely to render the compound immunogenic to an animal, or likely to give rise to secondary toxic effects, or likely to decrease the maximal tolerated dose are preferably avoided. Moreover, the substituents are preferably chosen in order to improve solubility of the compound, especially the solubility in body fluids of an animal.

For determining preferred substituents in the formula (a), (a′) and (b), the criteria to be applied are the following, classified by decreased significance:
to confer an enhanced anti-proliferative activity to the whole compound; or
to confer an anti-proliferative activity more precisely targeted on tumour cells to the whole compound; or
to confer to the compound a less immunogenic or toxic character, or
to confer a better solubility to the whole compound or
to confer a metabolic protection to the compound thus increasing the bioavailability or the half-life, or
having a smaller molecular weight.

Different structures are preferred in the context of the present invention, namely R₁ is preferably an oxygen atom, in any of the formula (a), (a′) or (b), or NH.

Independently of the nature of R₁ moiety, the R₂ moiety is preferably a methyl in any of the formula (a), (a′) or (b). Preferably, R₁ is O and R₂ is CH₃ or R₁ is NH and R₂ is CH₃.

Compounds according to this aspect of the invention have advantageously at least one of cycles (I) and (II) which is substituted. Suitable substituents particularly preferred in the context of this invention are substituents capable of increasing the antiproliferative activity of the compound, substituents capable of increasing the solubility of the compound, substituents capable of decreasing the antigenic action of the compound and very importantly substituents conferring a metabolic protection to the compound. Such potential substituents are known from the skilled man.

Preferably, cycle (I) alone, cycle (II) alone, or cycles (I) and (II) are substituted. Cycle (III) may also be substituted. Preferably, each of the cycles (I), (II) and (III) possesses only one additional substitution, in addition to the substitution already present in formula (a), (a′) and (b). Preferred substituents for cycles (I) and (II) are fluorine (F) and (CH₂)ₘ—R₉, with m=1 or 2 and R₉] is

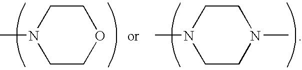

Such a substituent may also be adopted for cycle (III).

According to another preferred embodiment of the invention, Y represents a hydrogen atom in formula (a) or (b).

According to other preferred embodiments of the invention, R₁ is 0 and/or -R₂ is —CH₃, in any of the formula (a), (a′) and (b).

According to another embodiment, R₁ is N—(CH₂)ₚ—CO—N-methylpiperazine, preferably N—CO—N-methylpiperazine.

According to still another preferred embodiment R₁₆ is H or CH₃, and most preferably R₁₆ is H.

According to still another embodiment, R₁₇ is a furan, a thiophene, a pyrrole, a thiazole, a pyrrazole or an oxazole moiety, R₁ being in this case not O; preferably R₁₇ is a furan moiety.

Preferred compounds of the invention correspond to compounds wherein in formula (a) R₁ is O, —R₂ is —CH₃, R₃ is O, Y is H, R is NH—R₅ and R₅ is an aryl, a cycloalkyl, an heterocylcoalkyl, an heteroaryl or an arylakyl, preferably R₅ is a phenyl or R₅ is a benzyl or -R₅ is —C₆H₄—N(CH₃)₂. Other preferred compounds of the invention are those wherein in formula (a) R₁ is O, -R₂ is —CH₃, R₃ is NH, Y is H and

### R] is

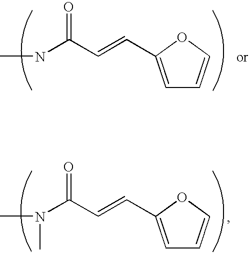

the furan moiety being optionally substituted with an alkyl having from 1 to 4 carbon atoms, NH₃, O, OH, CO—NH₂, O—CH₃, CO—OH, CO—OCH₃ or O—CO—CH₃. Alternatively, the furan moiety may be replaced by another heterocycloalkyl, for instance a thiophene, a pyrrole, a thiazole, a pyrrazole or an oxazole moiety, optionally substituted as mentioned above.

Optionally, R₁ may be NH instead of O,

Still other preferred compounds according to this first aspect of the invention are compounds, wherein in formula (b): R₁ is O, -R₂ is —CH₃, Y is H and R₄] is

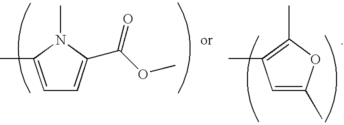

Other compounds preferred according to the present invention are compounds wherein in formula (a):

### R₁ is O or NH, -R₂ is —CH₃, R₃ is NH, Y is H and -R is —NH—CO—C₆H₅.

Other molecules which are preferred in the context of the present invention have the general formula (a) with the following features: R₁ is O, R₂ is H or CH₃, R₃ is S, Y is H,

R is NH—(CH₂)ₙ—R₁₂, n=0 and R₁₂] is

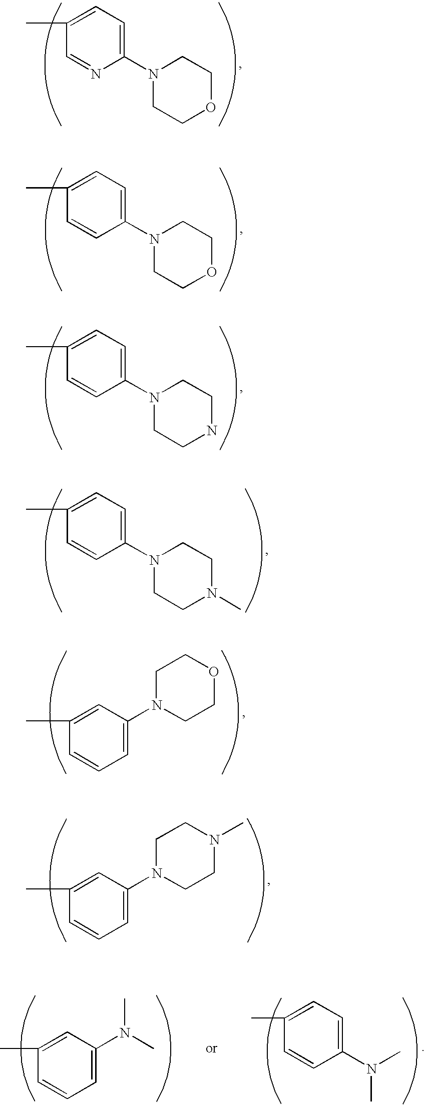

Optionally, in the preceding formula, R₃ may be O instead of S.

Still other molecules also having a structure corresponding to the general formula (a) and which are particularly preferred in the context of this invention have the following features: R₁ is O, -R₂ is —CH₃, R₃ is S, Y is H, R is NH—(CH₂)ᵣ—R₁₂, n=0 and R₁₂ is C₆H₅. Optionally, R₃ may be O instead of S.

Another class of preferred compounds correspond to formula (a) wherein R₁ is O, -R₂ is —CH₃, R₃ is S, Y is H, R is NH—CO—R₈ and -R₈ is —CH₂—CH₂—C₄H₃O or
R₈] is

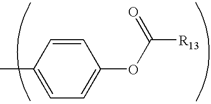

wherein R₁₃ is an alkyl in C₁ to C₆.

Alternatively, other preferred compounds of the invention are compounds, wherein in formula (a):

R₁ is O, -R₂ is —CH₃, R₃ is S, R is NH—(CH₂)ₙ—R₇, n=1 and R₇ is —C₆H₅.

For compounds of the invention having the general formula (a′), preferred substituents are the following: R₁ is O, -R₂ is —CH₃, R₃ is S—CH₃ and R] is

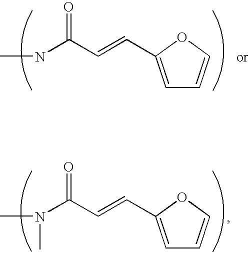

the furan moiety being optionally substituted with an alkyl having from 1 to 4 carbon atoms, NH₃, O, OH, CO—NH₂, O—CH₃, CO—OH, CO—OCH₃ or O—CO—CH₃.

Alternatively, the furan moiety may be replaced by another heterocycloalkyl, especially a thiophene, a pyrrole, a thiazole, a pyrrazole or an oxazole moiety, optionally substituted as mentioned above.

### Examples of particularly preferred compounds are

Compound 03: 1-[3-methoxy-4-(2-oxo-2H-chromen-3-yl)phenyl]-3-phenylurea,
Compound 04: 1-[3-methoxy-4-(2-oxo-2H-chromen-3-yl)phenyl]-3-phenylthiourea,
Compound 05: 4-fluoro-N-({[3-methoxy-4-(2-oxo-2H-chromen-3-yl)phenyl]amino}carbonothioyl)benzamide,
Compound 06: 1-[4-(dimethylamino)phenyl]-3-[3-methoxy-4-(2-oxo-2H-chromen-3-yl)phenyl]urea,
Compound 07: N-(imino{[3-methoxy-4-(2-oxo-2H-chromen-3-yl)phenyl]amino}methyl)benzamide,
Compound 08: 1-[3-methoxy-4-(2-oxo-2H-chromen-3-yl)phenyl]-3-(6-morpholin-4-ylpyridin-3-yl)thiourea,
Compound 09: methyl 5-({[3-methoxy-4-(2-oxo-2H-chromen-3-yl)phenyl]amino}sulfonyl)-1-methyl-1H-pyrrole-2-carboxylate,
Compound 10: methyl N-[(2E)-3-(2-furyl)prop-2-enoyl]-NA-[3-methoxy-4-(2-oxo-2H-chromen-3-yl)phenyl]-N-methylimidothiocarbamate,
Compound 12: (2E)-3-(2-furyl)-N-(imino{[3-methoxy-4-(2-oxo-2H-chromen-3-yl)phenyl]amino}methyl)acrylamide,
Compound 13: N-[3-methoxy-4-(2-oxo-2H-chromen-3-yl)phenyl]-2,5-dimethylfuran-3-sulfonamide,
Compound 14: methyl N-[(2E)-3-(2-furyl)prop-2-enoyl]-Ar-[3-methoxy-4-(2-oxo-2H-chromen-3-yl)phenyl]imidothiocarbamate,
Compound 15: (2E)-3-(2-furyl)-N-({[3-methoxy-4-(2-oxo-1,2-dihydroquinolin-3-yl)phenyl]amino}carbonothioyl)acrylamide,
Compound 16: methyl N-(4-fluorobenzoyl)-Ar-[3-methoxy-4-(2-oxo-2H-chromen-3-yl)phenyl]-N-methylimidothiocarbamate,
Compound 17: (2E)-3-(2-furyl)-N-({[3-methoxy-4-(1-methyl-2-oxo-1,2-dihydroquinolin-3-yl)phenyl]amino}carbonothioyl)acrylamide,
Compound 18: 1-[3-(dimethylamino)phenyl]-3-[3-methoxy-4-(2-oxo-2H-chromen-3-yl)phenyl]urea,
Compound 19: N′-((E)-3-furan-2-yl-acryloyl)-N-[3-methoxy-4-(2-oxo-2H-chromen-3-yl)-phenyl]-N-methyl-guanidine,
Compound 20: 1-[3-methoxy-4-(2-oxo-2H-chromen-3-yl)-phenyl]-3-(4-morpholin-4-yl-phenyl)-thiourea,
Compound 21: 1-((E)-3-furan-2-yl-acryloyl)-3-{4-[1-(2-hydroxy-ethyl)-2-oxo-1,2-dihydro-quinolin-3-yl]-3-methoxy-phenyl}-thiourea,
Compound 22: N-Benzoyl-N′-[3-methoxy-4-(2-oxo-1,2-dihydro-quinolin-3-yl)-phenyl]-guanidine,

Compound 23: 2,5-dimethyl-furan-3-sulfonic acid acetyl-[3-methoxy-4-(2-oxo-2H-chromen-3-yl)-phenyl]-amide,
Compound 24: 1-((E)-3-furan-2-yl-acryloyl)-3-[3-methoxy-4-(2-oxo-2H-chromen-3-yl)-phenyl]-1,2-dimethyl-isothiourea, and
Compound 25: 1-(4-fluoro-benzoyl)-3-[3-methoxy-4-(2-oxo-2H-chromen-3-yl)-phenyl]-1,2-dimethyl-isothiourea.

According to a second aspect, the present invention concerns a compound having the general formula:

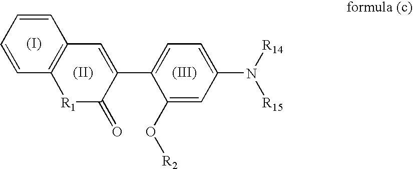

wherein the cycles (I), (II) and (III) may be optionally substituted on 1, 2, 3 or 4 of the available carbons with a group chosen amongst: Br, Cl, F, I, OH, O—CH₃, COOH, and (CH₂)ₘ—R₉, wherein R₉ represents H, CH₃, OH, O—CH₃, COOH, NH₃, CO—NH₂, NH—CO-phenyl, an alkyl, an heteroaryl or an heterocycloalkyl and m=1, 2, 3 or 4;
wherein R₁ represents O, NR₁₆ with R₁₆ being H or an alkyl having from 1 to 4 carbon atoms, or N—(CH₂)ₚ—R₇, with R₇ representing OH, CCOR₁₆, CO—NHR₁₆, NH₂R₁₆, O—CH₂R₁₆, CN, O—CO—CH₂R₁₆, a cycloalkyl, a heterocycloalkyl, an aryl or a heteroaryl, possibly substituted with one or more of the followings radicals: an alkyl, an aryl, a heteroaryl, a cycloalkyl, a heterocycloalkyl, F, Cl, NH₂, C≡N, OH, CO—NH₂, O—CH₃, CO—OH, CO—OCH₃ or O—CO—CH₃, and p=0, 1, 2, 3, 4, 5 or 6 and with R₁₆ being H or an alkyl with 1, 2, 3 or 4 carbon atoms;
wherein R₂ represents H or an alkyl with less than 4 carbon atoms,
wherein R₁₄ and R₁₅ are, independently, any type of substituent with a molecular weight less than 400 Dalton, provided that R₁₄ and R₁₅ are not simultaneously oxygen,
   said compound being for use in therapy.

The invention is also directed to base and acid addition salts of said compounds, the geometric isomers, enantiomers and diastereoisomers thereof, also for use in therapy.

Each substitutent is chosen independently of the others.

As for the compounds according to the first aspect, the substituents are preferably chosen in order to confer an enhanced anti-proliferative activity to the whole compound; or to confer an anti-proliferative activity more precisely targeted on tumour cells to the whole compound; or to confer to the compound a less immunogenic character, or to confer a better solubility to the whole compound (especially the solubility in body fluids of an animal), or to confer a metabolic protection to the compound thus increasing the bioavailability, or to reduce the molecular weight of the whole compound.

The overall molecular mass of the compound is preferably less than 1000 Da. Preferably, the molecular weight is less than 800 Da, preferably less than 700, 650 or 600 Da. Indeed, the compound is preferably capable of passing (passively) the cell membrane. Therefore, the molecular weight of the compound is if possible minimized.

The use of such compounds in therapy may be for prophylactic and/or for curative purposes. Preferably, a compound according to this second aspect of the invention has anti-proliferative properties. The compounds have preferably anti-proliferative (or cytostatic) effect mainly targeted on tumour cells, cancerous cells or immortalized cells. For example, after contacting a population of cells with a compound, the decrease of the number of cells is 10% greater on a population of tumour or cancerous cells than on a population of normal cells. The difference in the effect on a population of tumour or cancerous cells with a population of normal cells is preferably at least 20%, at least 30% or at least 50% or even more.

More preferably, said compounds have cytotoxic activity or pro-apoptotic activity. Preferably, this activity is preferentially targeted on tumour cells. Similarly, this means that the cytotoxic or pro-apoptotic effect is at least 10% more intense on a population of tumour or cancerous cells than on a population of normal cells.

The criteria governing the choice of preferred substituents are the same as for the first aspect of the invention.

The compounds as defined are useful for eliminating cancerous cells; they are thus very useful in the treatment of cancers, leukaemia or any type of tumours.

The compounds according to this second aspect of the invention are for use in human or veterinarian therapy and are to be administered to a patient, which is an animal, preferably a mammal and most preferably a human being. Therefore, substituents likely to render the compound immunogenic to an animal, or likely to give rise to secondary toxic effects, or likely to decrease the maximal tolerated dose, are preferably avoided. Preferred patients are mammals, especially farm animals and pets, and humans (children or adults).

Different structures are preferred in the second aspect of the present invention, namely R₁ is preferably an oxygen atom in formula (c), or NH.

Independently of the R₁ moiety, the R₂ moiety is preferably a methyl. Preferably, R₁ is O and R₂ is CH₃ or R₁ is NH and R₂ is CH₃.

Compounds according to this aspect of the invention have advantageously at least one of cycles (I) and (II) which is substituted. Suitable substituents particularly preferred in the context of this invention are substituents capable of increasing the antiproliferative activity of the compound, substituents capable of increasing the solubility of the compound, substituents capable of decreasing the antigenic action of the compound and importantly substituents conferring a metabolic protection to the compound. Such potential substituents are known from the skilled man.

Preferably, cycle (I) alone, cycle (II) alone, or cycles (I) and (II) are substituted. Cycle (III) may also be substituted. Preferably, each of the cycles (I), (III) and (III) possesses only one additional substitution, in addition to the substitution already present in formula (c).

Preferred substituents for cycles (I) and (II) are fluorine (F) and (CH₂)ₘ—R₉, with m=1 or 2 and -R₉ is an aryl, a cycloalkyl, an heterocylcoalkyl, an heteroaryl or an arylakyl, and preferably, R₉] is

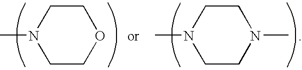

According to another preferred embodiment of the invention, Y represents a hydrogen atom.

According to other preferred embodiments of the invention, R₁ is O and/or -R₂ is —CH₃.

In a preferred compound of the invention, at least one of R₁₄, R₁₅, NR₁₄ and NR₁₅ contains an amide, a sulphonamide, a ketone, a furan, a thiophene, a pyrrole, a thiazole, a pyrrazole, an oxazole, a urea, a thiourea group, a benzyl, a phenyl, an acyl or a guanidine.

It is preferred that R₁₄ and R₁₅ are different.

According to another preferred embodiment, at least one of R₁₄ and R₁₅ is H or CH₃.

It is to be noted that according to the general formula (c), R₁₄ and R₁₅ are linked to the nitrogen via a single bond. However, double bound can also be envisaged, provided that R₁₄ and R₁₅ are not simultaneously oxygen atoms.

It is preferred that R₁₆ is H or CH₃.

According to another embodiment, R₁ is N—(CH₂)ₚ—CO—N-methylpiperazine, preferably N—CO—N-methylpiperazine.

Preferred compounds of this second aspect are those exemplified according to the first aspect of the invention, i.e. compounds 03, 04, 05, 06, 07, 08, 09, 10, 12, 13, 14, 15, 16, 17, 18, 19, 20, 21, 22, 23, 24 and 25.

Other preferred compounds according to this second aspect of the invention are:
Compound 01: (2E)-3-(2-furyl)-N-({[3-methoxy-4-(2-oxo-2H-chromen-3-yl)phenyl]amino}carbonothioyl)acrylamide and
Compound 02:
N-({[3-methoxy-4-(2-oxo-2H-chromen-3-yl)phenyl]amino}-carbonothioyl)benzamide.

In the following, a compound of the invention is a reference to a compound according to either the first or the second aspect of the invention.

The present invention is also directed to salts of the compounds of the invention, e.g. chlorhydrate salts, especially pharmaceutically acceptable salts of the compounds of the invention.

Moreover, the invention also concerns metabolites of the compounds of the invention, especially metabolites formed in vivo. Furthermore, the invention also relates to prodrugs of the compounds of the invention, which are transformed in vivo into said compounds.

Importantly, it is to be noted that “a compound of the invention” encompasses any stereoisomeric and/or tautomeric form thereof, as also mixtures of these different forms.

As mentioned above, a compound of the invention has preferably cytotoxic or cytostatic properties on tumor cells. Preferred tumoral cells are lung, prostate, breast, colon, melanoma, lymphoma and/or myeloid tumor cells.

As mentioned above, a compound of the invention is preferably soluble in water and in different body fluids. With respect to the solubility in water, a compound of the invention can preferably be dissolved in water at a molarity above 50.10⁻⁶ mol·L⁻¹, preferably at molarity above 500.10⁻⁶ mol·L⁻¹ or even more preferably above 800.10⁻⁶ mol·L⁻¹ or around 1 mmol·L⁻¹ (i.e. around 10⁻³ mol·L⁻¹).

According to a preferred embodiment of the present invention, a compound as defined above is capable of interacting, directly or indirectly with the anti-apoptotic protein Bcl-xL. It is preferred that a compound of the invention binds the protein Bcl-xL, at least in vitro, preferably with a Kd less than 1 μM, preferably less than 100 nM, or even less, for example less than 20 nM or less than 10 nM. Such compounds are particularly preferred.

A suitable protocol for defining the Kd of the interaction between a molecule and the protein Bcl-xL is given in the experimental section of this application (see example 5).

The present invention also concerns a pharmaceutical formulation comprising a compound of the invention, such that the bioavailability of the compound is at least 20%, preferably at least 50%, even preferably at least 80%, 90% or 95%.

The present invention also concerns a composition comprising a compound as defined according to the first or the second aspect, in association with at least one other active ingredient. Said other active ingredient may be a therapeutic agent of any type, for example an anti-inflammatory agent or a compound counterbalancing any side-effect of the compound of the invention (reducing, abolishing or delaying said potential side-effects).

Advantageously, the compound of the invention is used or provided in association with at least one other anti-tumor agent or chemotherapeutic or with radiation. It has indeed been observed by the inventors that the anti-cancer effect of a compound of the invention is enhanced when combined with another anti-cancer treatment. A composition may advantageously comprise an association of a compound of the invention with at least two different other chemotherapeutics, or at least three or more.

Suitable anti-tumor agents which can be combined with a compound according to the invention are for example: taxol (paclitaxel), etoposide, doxorubicin, cis-platin, retinoic acid, tamoxifen, vinblastin, vincristin, cyclophosphamide, topotecan, irinotecan, gemcitabine or rapamycin. This list is however not exhaustive, any other chemotherapeutic may be combined with a compound of the invention.

Preferably, such a composition comprises a compound of the invention and another chemotherapeutic having also anti-proliferative property, i.e. having cytostatic and/or cytotoxic activity on tumour cells.

The invention also relates to the use of a composition as mentioned above for the manufacture of a medicament to be administered to a subject for treating cancer or any other pathology implying the abnormal growth of tissue resulting from uncontrolled, progressive multiplication of cells or the unwanted or pathological sustained proliferation of cells. The subject may be an animal, especially a mammal, and is preferably a human patient.

The invention is also directed to a product containing a compound according to the invention and a further active agent, preferably an anti-tumor agent, as a combined preparation for simultaneous, separate or sequential use in therapy. The compound of the invention and the other anti-tumor agent are preferably the active ingredients of the product.

A combined preparation for simultaneous therapy is defined as a treatment comprising administration of a compound of the invention and a further agent, preferably an anti-tumor agent, at the same time. Said compound and agent can be packaged together or as two separate entities to be given simultaneously.

A combined preparation for sequential therapy is defined as a treatment comprising the successive administration of a compound of the invention alone followed by the administration of a further agent alone, preferably an anti-tumor agent, or in the reverse order.

A combined preparation for separate therapy is defined as a treatment comprising administration of a compound of the invention and administration of a further agent, preferably an anti-tumor agent, wherein both therapies are concomitant, the administrations being however not all made simultaneously.

The present invention also concerns a pharmaceutical composition comprising a compound according to the invention, i.e. according to the first or the second aspect of the invention, and a pharmaceutically acceptable carrier. Contrary to an active ingredient, a pharmaceutically acceptable carrier is an excipient, devoid of therapeutic activity by itself. The skilled man in the pharmaceutical domain is able to define potential pharmaceutically acceptable carriers to be combined with a compound of the invention, depending on the chosen mode of administration. Different carriers may be envisaged and incorporated together in the pharmaceutical composition. Moreover, a compound of the invention can be combined with more than one acceptable carrier, for example with at least two or three, or more. Other ingredients may be added in a pharmaceutical composition of the invention, e.g. flavours, dyes, which are active or inactive from a therapeutic point of view.

As mentioned above, preferred additional ingredients are other anti-cancer agents and especially anti-cancer agents having anti-proliferative activity, for example at least one other compound of the invention.

According to a preferred embodiment of the invention, the pharmaceutical composition contains a compound of the invention, a suitable carrier and taxol. Respective proportions of each ingredient are to be determined by the skilled man in the domain.

A pharmaceutical composition of the invention can be designed for an administration via intralesional, intraperitoneal, intramuscular or intravenous injection; via infusion; liposome-mediated delivery; topical, nasal, oral, anal, subcutaneous, vaginal, sublingual, uretheral, transdermal, intrathecal, ocular or otic delivery.

The mode of administration is to be determined depending on the pathology to be treated. Indeed, a different mode of administration is to be envisaged for treating for example a solid tumour and metastases.

The present invention is also directed to a compound of the invention for the manufacture of a medicament to be administered to a subject for treating cancer or other pathologies characterized by the abnormal growth of tissue resulting from uncontrolled, progressive multiplication of cells or by unwanted or pathological sustained proliferation of cells.

The subject to be treated is preferably a mammal, especially cats, dogs, horses and humans. Particularly preferred patients are humans.

According to this embodiment of the invention, the medicament to be prepared is to be administered via intralesional, intraperitoneal, intramuscular or intravenous injection; via infusion; liposome-mediated delivery; topical, nasal, oral, anal, subcutaneous, vaginal, sublingual, uretheral, transdermal, intrathecal, ocular or otic delivery. However, an alternative mode of administration may also be envisaged.

The invention also relates to the use of a compound of the invention for the manufacture of a medicament to be administered simultaneously, separately or sequentially with at least one other active agent, preferably an anti-tumor agent or a chemotherapeutic, to a subject for treating cancer or other pathologies characterized by the abnormal proliferation of cells. As mentioned above, the invention is not limited to the addition of only one other anti-tumor agents and several different agents can enter in the composition of a medicament as defined. Examples of potential other anti-tumor agents are: taxol (paclitaxel), etoposide, doxorubicin, cis-platin, retinoic acid, tamoxifen, vinblastin, vincristin, cyclophosphamide, topotecan, irinotecan, gemcitabine or rapamycin. This list is however not exhaustive.

The invention also concerns a process for inhibiting cell proliferation comprising contacting a cell with a compound of the invention (first or second aspect). Such a process can be carried out in vivo or ex vivo, but is preferably carried out in vitro.

### EXAMPLES

### Example 1

### Synthesis of Compounds of the Invention

### 1.A Synthesis of Courmarine Derivatives

The main steps and conditions to be applied are summarized in the following reaction scheme. ¹R and ²R are potential substituents of cycles (I) and (III) in formula (a), (a′), (b) and (c) as defined above. ³R, ⁶R and ⁷R represent potential substituents in order to obtain compounds according to formula (a), (a′), (b) or (c) as defined above.

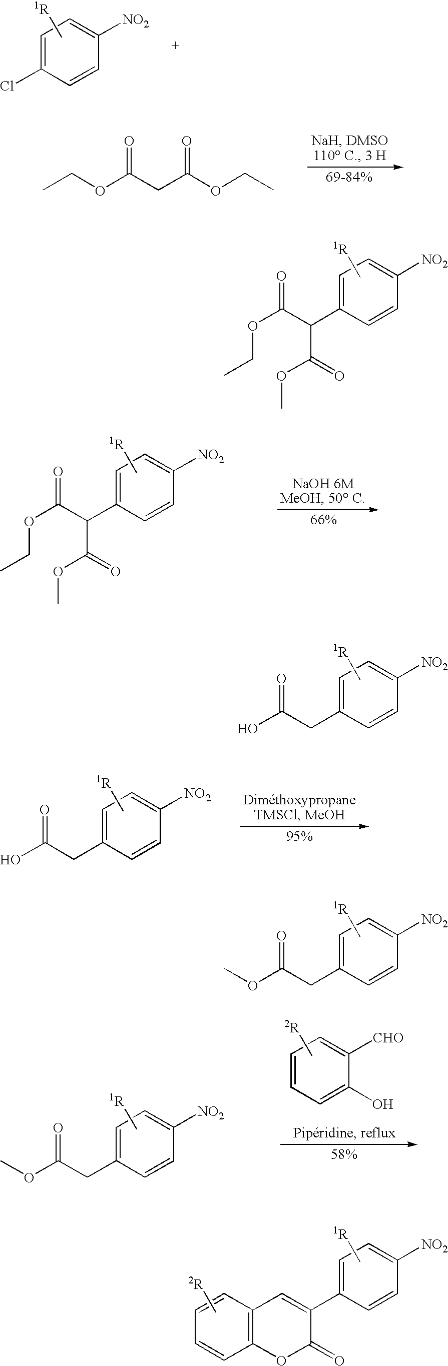

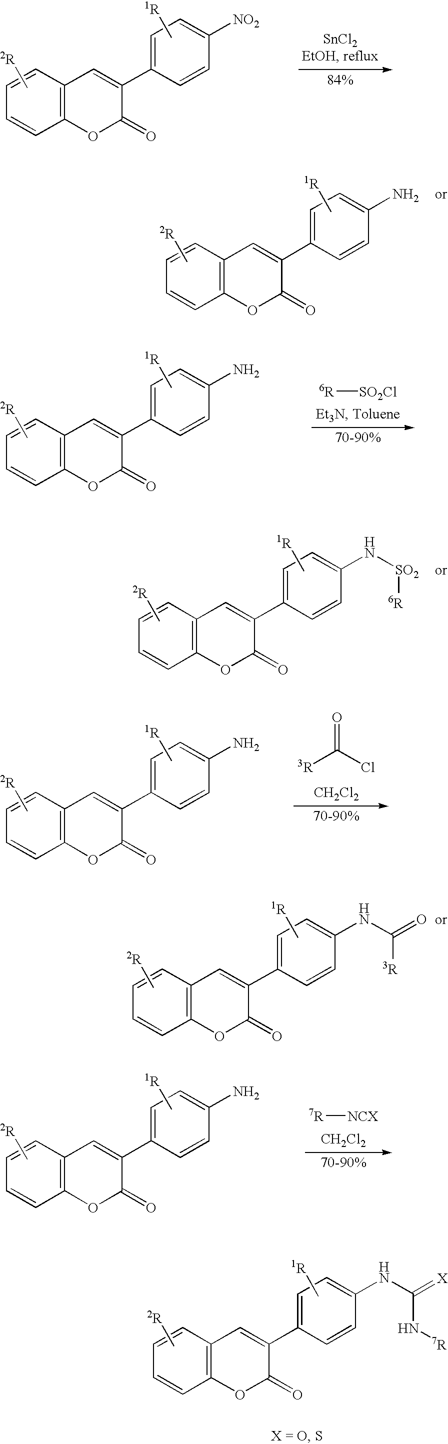

More specifically:

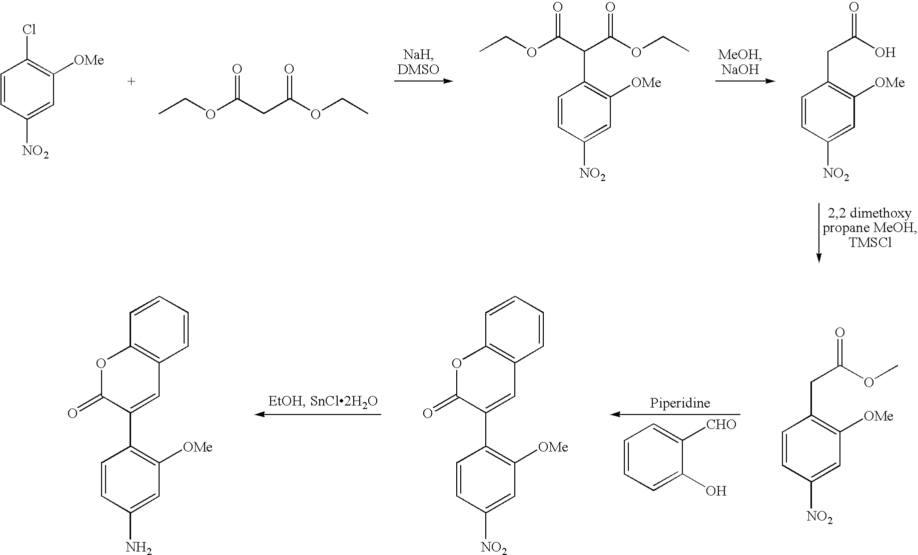

### Step 1: Preparation of 2-(2-methoxy-4-nitro-phenyl)-malonic acid diethyl ester

Diethyl malonate (253 mmol) was slowly added at room temperature to a suspension of NaH (60% in oil, washed with cyclohexane) (275 mmol) in DMSO (140 ml). The reaction was stirred for 20 minutes before addition of 2-chloro-5-nitroanisole (105.5 mmol). After 5 hours of stirring at 110° C., the solution was poured onto water (1.5 l) then acidified to pH=1 by addition of concentrated HCl. The solution was stirred for another 20 minutes. The reaction mixture was filtered and the solid obtained, washed with water and dried yielding 27.1 g of a red solid.

The residue was triturated in ice-cold methanol, filtered, washed with cold methanol and dried to yield 19.4 g of the desired product.

### Step 2: Preparation of (2-methoxy-4-nitro-phenyl)-acetic acid

The substrate (62.3 mmol) (prepared in Step 1) was dissolved in methanol (50 ml) and cooled with an ice bath. To this solution was added a solution of 6M NaOH (31 ml) during a 15 minute period and the resulting mixture was stirred for an additional 5 minutes at 0° C. The reaction mixture was then warmed to 50° C. and stirred at this temperature for 90 minutes.

The methanol was removed under reduced pressure. The residue was cooled to 0° C. and acidified to pH=1 by addition of concentrated HCl. The reaction mixture was filtered and the solid washed with water. Ethyl acetate was added to the filtrate and the biphasic solution extracted. The organic layer was dried over magnesium sulfate, filtered and evaporated under reduced pressure to yield 9.4 g of the desired product.

### Step 3: Preparation of (2-methoxy-4-nitro-phenyl)-acetic acid methyl ester

The substrate (39.3 mmol) (prepared in Step 2) was dissolved in a mixture of methanol (45 ml) and 2,2-dimethoxypropane (16 ml). Chlorotrimethylsilane (0.5 ml) was added and the solution was stirred at room temperature for 2 hours.

The solvents were removed under reduced pressure. The residue was dissolved in ethyl acetate, washed with NaHCO₃, water and saturated aqueous sodium chloride. The organic phase was dried over magnesium sulfate, filtered and evaporated under reduced pressure to yield 8.4 g of an orange solid.

### Step 4: Preparation of 3-(2-methoxy-4-nitro-phenyl)-chromen-2-one

Salicylaldehyde (29 mmol) was added at room temperature to a solution of substrate (19.5 mmol) (prepared in Step 3) in piperidine (20 ml). The reaction mixture was stirred under reflux conditions for 4 hours. At the end of the reaction, the solution is cooled to 0° C. and ethanol was added. The precipitate was filtered and washed with ethanol to yield 3.6 g of the title compound as a yellow powder.

### Step 5: Preparation of 3-(4-amino-2-methoxy-phenyl)-chromen-2-one

To a solution of substrate (12.2 mmol) (prepared in Step 4) in ethanol (40 ml) was added tin (II) chloride dihydrate (61 mmol). The resulting reaction mixture was stirred under reflux conditions for 2.5 hours. The solution was allowed to cool to room temperature before addition of NaOH 1M and stirred for 1 hour.

The mixture was filtered on celite and washed with ethanol. The ethanol was removed under reduced pressure and the resulting solution extracted with ethyl acetate (3×). The organic layers were combined and washed with NaOH 1M, water and saturated aqueous sodium chloride. The organic phase was dried over magnesium sulfate, filtered and evaporated under reduced pressure to yield 2.8 g of a yellow powder.

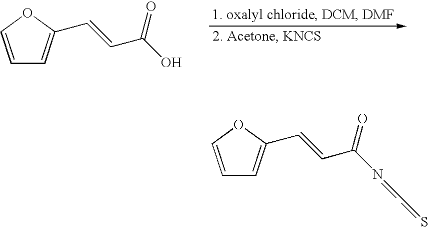

### Step 6: Preparation of (E)-3-furan-2-yl-acryloyl isothiocyanate

3-(2-furyl)acrylic acid (obtained from Aldrich; 72.4 mmol) was dissolved in DCM (50 ml). To this solution were successively added oxalyl chloride (145 mmol) and DMF (0.5 ml). The reaction was stirred at room temperature for 1 hour. The solvents were evaporated under reduced pressure and azeotroped with toluene. The residue was dissolved in acetone (50 ml). To this solution was added potassium thiocyanate (145 mmol) and the solution was stirred for an additional 30 minutes. The solvents were evaporated and the residue was purified by flash column chromatography (100% DCM) to yield 10.6 g of yellow oil.

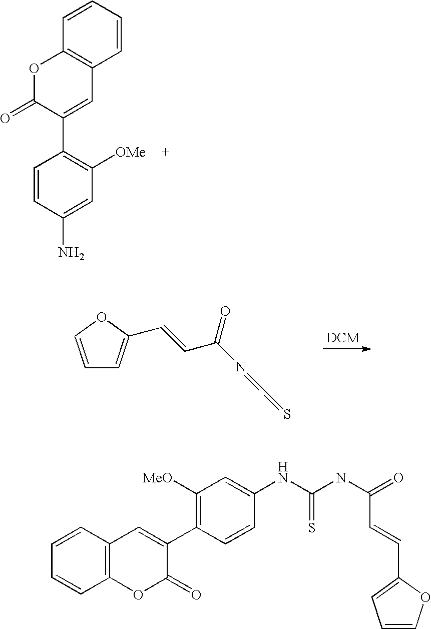

### 1.A.1: Preparation of (2E)-3-(2-furyl)-N-({[3-methoxy-4-(2-oxo-2H-chromen-3-yl)phenyl]amino}carbonothioyl)acrylamide (Compound 01)

### Alternative Name: 1-((E)-3-furan-2-yl-acryloyl)-3-[3-methoxy-4-(2-oxo-2H-chromen-3-yl)-phenyl]-thiourea

3-(4-amino-2-methoxy-phenyl)-chromen-2-one (6 mmol) (prepared in Step 5) and (E)-3-furan-2-yl-acryloyl isothiocyanate (24 mmol) (prepared in Step 6) were dissolved in DCM (50 ml). The reaction was stirred at room temperature for 2 hours. The solvents were evaporated under reduced pressure. The residue was dissolved in ethanol and the solution was stirred for an additional 30 minutes. The solution was filtered and the solid washed to yield 2.6 g of a yellow powder (mp 227-230° C.). LCMS (M+H⁺=447, 100%), NMR (250 MHz, DMSO): δ 12.89 (br s, 1H), 11.72 (br s, 1H), 8.07 (s, 1H), 7.93 (s, 1H), 7.80-7.30 (m, 8H), 7.01 (d, J 3.5, 1H), 6.82 (d, J 15.5, 1H), 6.70-6.67 (m, 1H), 3.78 (s, 3H).

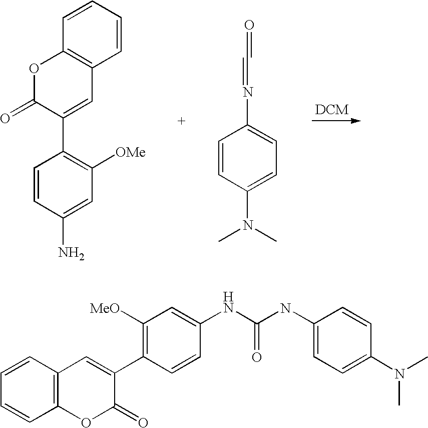

### 1.A.2: Preparation of 1-(4-dimethylamino-phenyl)-3-[3-methoxy-4-(2-oxo-2H-chromen-3-yl)-phenyl]-urea Hydrochloride Salt (Compound 06 Hydrochloride Salt)

3-(4-amino-2-methoxy-phenyl)-chromen-2-one (12.8 mmol) (prepared in Step 5) and 4-(Dimethylamino)phenyl isocyanate (15.4 mmol) were dissolved in DCM (35 ml). The reaction was stirred at room temperature for 6 days. The solvents were evaporated under reduced pressure. The residue was triturated with diethyl ether, filtered and the solid washed with diethyl ether to yield 5.6 g of solid. This solid was dissolved in methanol. A solution of hydrogen chloride in methanol was added ant the resulting mixture was stirred for 20 minutes. The solvents were evaporated under vacuum. The residue obtained was washed with diethyl ether to yield 5.7 g of a light beige powder. LCMS (M+H⁺=430, 100%), NMR (250 MHz, DMSO): δ 9.43-9.30 (m, 2H), 8.00 (s, 1H), 7.73 (d, J 7.8, 1H), 7.65-7.30 (m, 8H), 7.25 (d, J 8.0, 1H), 7.01 (dd, J 8.2 and 1.7, 1H), 3.74 (s, 3H), 3.16 (s, 6H).

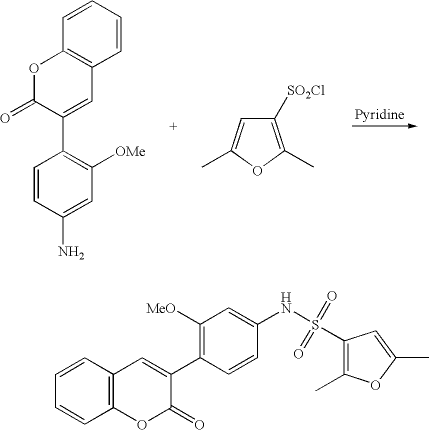

### 1.A.3: Preparation of N-[3-methoxy-4-(2-oxo-2H-chromen-3-yl)phenyl]-2,5-dimethylfuran-3-sulfonamide (Compound 13)

### Alternative Name: 2,5-dimethyl-furan-3-sulfonic acid-3-[3-methoxy-4-(2-oxo-1,2-dihydro-quinolin-3-yl)-phenyl]-amide

3-(4-amino-2-methoxy-phenyl)-chromen-2-one (5.6 mmol) (prepared in Step 5) and 2,5-dimethyl-furan-3-sulfonyl chloride (obtained by Maybridge; 6.2 mmol) were dissolved in pyridine (10 ml). The reaction mixture was stirred at room temperature for 12 hours. The solution was poured onto a solution of HCl 1M, extracted with DCM and washed with a solution of HCl 1M, water and a saturated solution of aqueous sodium chloride. The solvents were evaporated under reduced pressure. The residue was triturated with diethyl ether, filtered and washed with diethyl ether to yield 1.7 g of a beige powder. LCMS (M+H⁺=426, 100%), NMR (250 MHz, DMSO): δ 10.41 (br s, 1H), 7.98 (br s, 1H), 7.71 (dd, J 7.7 and 1.2, 1H), 7.61 (td, J 8.2 and 1.5, 1H), 7.45-7.25 (m, 2H), 7.23 (d, J 8.2, 1H), 6.85 (d, J 1.7, 1H), 6.75 (dd, J 8.2 and 2, 1H), 6.26 (s, 1H), 3.68 (s, 3H), 2.41 (s, 3H), 2.21 (s, 3H).

### 1.B Synthesis of Oxoquinoline Derivatives

¹R and ⁴R are potential substituents of cycles (I), (II) and (III) in formula (a), (a′), (b) and (c), as defined above. ⁵R represents potential substituents in order to obtain compounds according to formula (a), (a′), (b) or (c) as defined above.

The main steps and conditions to be applied are summarized in the following reaction scheme:

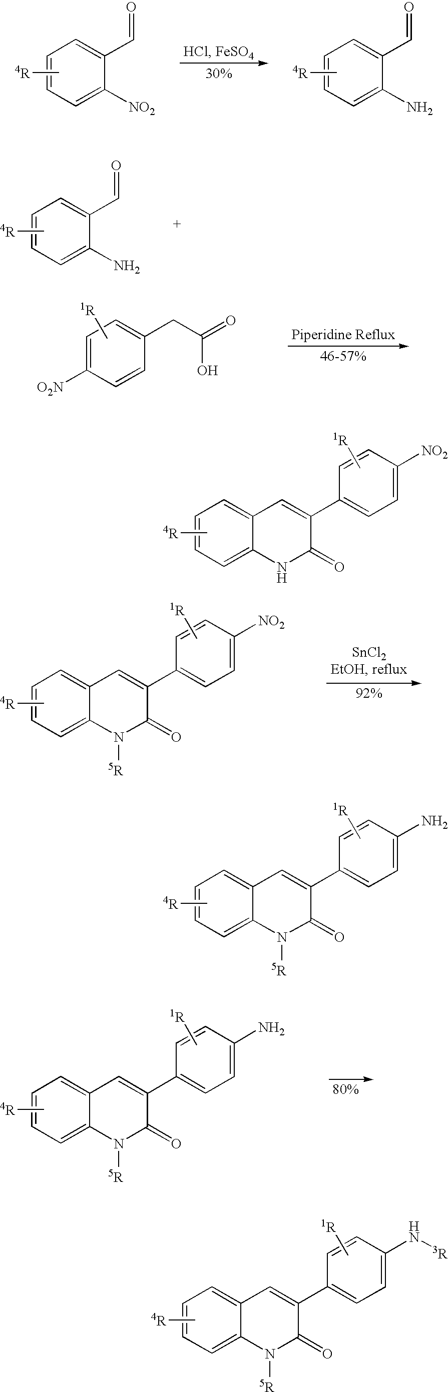

More specifically:

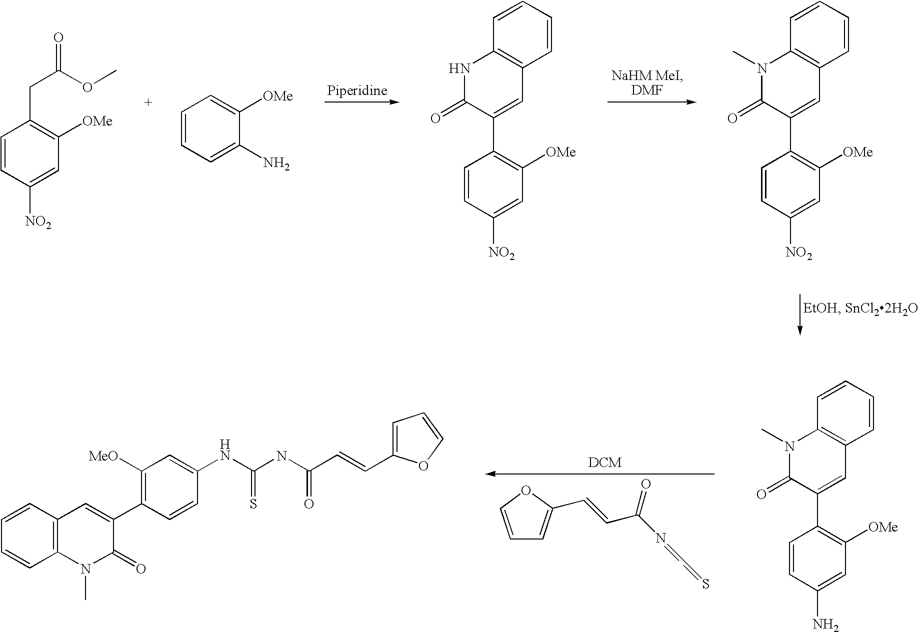

### Step 1: Preparation of 3-(2-methoxy-4-nitro-phenyl)-1H-quinolin-2-one

o-aminobenzaldehyde (prepared according to procedure described in Org. Synth., coll. Vol. 3, 56) (17 mmol) was added at room temperature to a solution of (2-methoxy-4-nitro-phenyl)-acetic acid methyl ester (15 mmol) (prepared in Step 3 of Example 1A) in piperidine (15 ml). The reaction mixture was stirred under reflux conditions for 2 hours. At the end of the reaction, the solution was cooled to room temperature and ethanol (50 ml) was added. The mixture was stirred for 30 minutes then cooled to 0° C. The precipitate was filtered to yield 2.2 g of the title compound as a yellow solid.

### Step 2: Preparation of 3-(2-methoxy-4-nitro-phenyl)-1-methyl-1H-quinolin-2-one

The substrate (7.4 mmol) (prepared in Step 1) was slowly added at 0° C. to a suspension of NaH (60% in oil, washed with cyclohexane) (14.8 mmol) in DMF (20 ml). The reaction was stirred for minutes at 0° C. before addition of methyl iodide (11.1 mmol). After 1 hours of stirring at 0° C., the solution was poured onto water (150 ml) and filtered. The residue was dissolved in EtOAc and dried over anhydrous magnesium sulfate. The solvent was removed under reduced pressure to yield 2.1 g of the desired product as a yellow solid.

### Step 3: Preparation of 3-(4-amino-2-methoxy-phenyl)-1-methyl-1H-quinolin-2-one

To a solution of substrate (6.8 mmol) (prepared in Step 2) in ethanol (100 ml) was added tin (II) chloride dihydrate (33.8 mmol). The resulting reaction mixture was stirred under reflux conditions for 2 hours. The solvents were removed under reduced pressure and the residue obtained, dissolved in ethyl acetate and washed with a saturated solution of aqueous sodium hydrogen carbonate and a saturated solution of aqueous sodium chloride.

The organic phase was dried over magnesium sulfate, filtered and evaporated under reduced pressure to yield 1.9 g of a yellow solid.

### 1B.1: Preparation of (2E)-3-(2-furyl)-N-({[3-methoxy-4-(1-methyl-2-oxo-1,2-dihydroquinolin-3-yl)phenyl]amino}carbonothioyl)acrylamide (Compound 17)

### Alternative Name: 1-((E)-3-furan-2-yl-acryloyl)-3-[3-methoxy-4-(1-methyl-2-oxo-1,2-dihydro-quinolin-3-yl)-phenyl]-thiourea

3-(4-amino-2-methoxy-phenyl)-1-methyl-1H-quinolin-2-one (6.8 mmol) (prepared in Step 3) and (E)-3-furan-2-yl-acryloyl isothiocyanate (13.7 mmol) (prepared in Step 6 of Example 1A) were dissolved in DCM (5 ml). The reaction was stirred at room temperature for 2 hours. The solvents were evaporated under reduced pressure. The residue was dissolved in ethanol and the solution was filtered to yield 2.2 g of a yellow solid. LCMS (M+H⁺=460, 99%), NMR (250 MHz, DMSO): δ 12.88 (br s, 1H), 11.71 (br s, 1H), 7.95-7.85 (m, 2H), 7.74 (d, J 7.7, 1H), 7.70-7.50 (m, 4H), 7.28-7.20 (m, 3H), 7.01 (d, J 3.5, 1H), 6.82 (d, J 15.5, 1H), 6.70-6.68 (m, 1H), 3.73 (s, 3H), 3.68 (s, 3H).

### 1.C: Methylation Conditions

Compound 14 was formed by the reaction of the compound formed in example 1.A.1 (compound 01) with K₂CO₃ (2 eq.), MeI (1.5 eq), in Acetone. The reaction was heated to reflux for 4 hours.

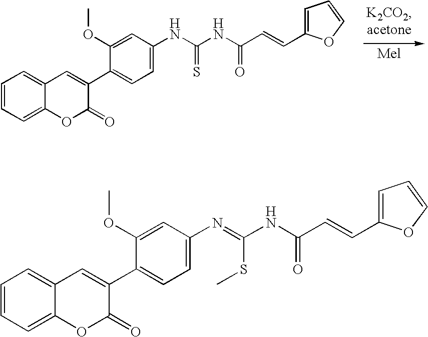

### 1.D

The characterization of representative compounds of the invention, obtained following the procedures of Examples 1.A, 1.B and 1.C and appropriate variations thereof, are reported below.

The LC_MS (Liquid Chromatography Mass Spectrometry) details were the following:

Automatic injector with an injection volume of 10 μl

### Column: THERMO HYPERSIL HYPERPREP RP C18, 8 μm, 150*4.6 mm

UV Detector: Agilent G1315A diode array detector [190400 nm]
ELSD (evaporative Light Scattering Detection) detector: Polymerlabs ELS2100, T° nebulizer=50° C., T° evaporator=90° C., gaz flow rate: 1.6 SLM

### Solvent Conditions

| Time | % | % | Flow rate |
|---|---|---|---|
| (min) | (Water + 0.05% TFA) | (ACN + 0.05% TFA) | (ml/min) |
| 0 | 80 | 20 | 1 |
| 8 | 0 | 100 | 1 |
| 8.10 | 0 | 100 | 1 |
| 13 | 0 | 100 | 1 |

### Column Regeneration Time: 7 min

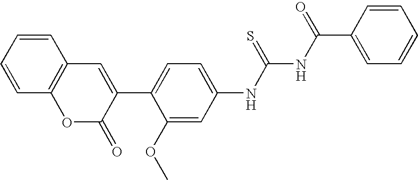

### Compound 02: N-({[3-methoxy-4-(2-oxo-2H-chromen-3-yl)phenyl]amino}carbonothioyl)benzamide

### Alternative Name: 1-Benzoyl-3-[3-methoxy-4-(2-oxo-2H-chromen-3-yl)-phenyl]-thiourea

NMR (250 MHz, DMSO): δ 12.75 (s, 1H), 11.65 (s, 1H), 8.08 (s, 1H), 8.02-7.95 (m, 2H), 7.80-7.35 (m, 10H), 3.78 (s, 3H).

LCMS 98% (431, M+1)

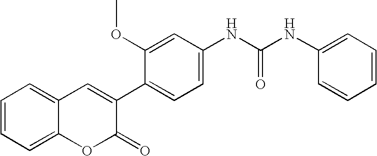

### Compound 03: 1-[3-methoxy-4-(2-oxo-2H-chromen-3-yl)-phenyl]-3-phenyl-urea

Pale yellow solid.

NMR (250 MHz, DMSO): δ 8.88 (s, 1H), 8.74 (s, 1H), 8.00 (s, 1H), 7.73 (d, J 7.7, 1H), 7.61 (t, J 8.5, 1H), 7.52-7.40 (m, 8H), 7.15-6.85 (m, 2H), 3.75 (s, 3H).

LC-MS purity: 100%.

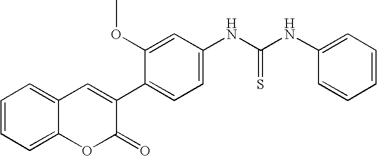

### Compound 04: 1-[3-methoxy-4-(2-oxo-2H-chromen-3-yl)-phenyl]-3-phenyl-thiourea

White solid.

NMR (250 MHz, DMSO): δ 9.94 (br s, 2H), 8.02 (s, 1H), 7.74 (dd, J 6.2 and 1.2, 1H), 7.62 (td, J 7.0 and 1.2, 1H), 7.50 (d, J 6.2, 2H); 7.45-7.25 (m, 6H), 7.20-7.10 (m, 2H), 3.74 (s, 3H).

LC-MS purity: 100%.

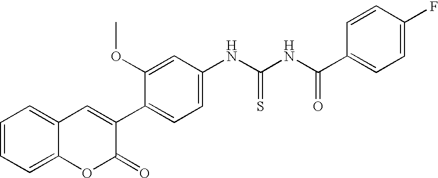

### Compound 05: 4-fluoro-N-({[3-methoxy-4-(2-oxo-2H-chromen-3-yl)phenyl]amino}carbonothioyl)benzamide

### Alternative Name: 1-(4-fluoro-benzoyl)-3-[3-methoxy-4-(2-oxo-2H-chromen-3-yl)-phenyl]-thiourea

White solid.

NMR (250 MHz, DMSO): δ 12.66 (br s, 1H), 11.70 (br s, 1H), 8.15-8.05 (m, 3H), 7.75 (d, J 8.2, 1H), 7.70-7.60 (m, 2H), 7.50-7.35 (m, 6H), 3.77 (s, 3H).

LC-MS purity: 100%.

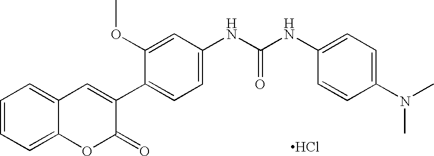

### Compound 06: 1-(4-dimethylamino-phenyl)-3-[3-methoxy-4-(2-oxo-2H-chromen-3-yl)-phenyl]-urea.HCl

Pale yellow solid.

NMR (250 MHz, DMSO): δ 9.43-9.30 (m, 2H), 8.00 (s, 1H), 7.73 (d, J 7.8, 1H), 7.65-7.30 (m, 8H), 7.25 (d, J 8.0, 1H), 7.01 (dd, J 8.2 and 1.7, 1H), 3.74 (s, 3H), 3.16 (s, 6H).

LC-MS purity: 98%

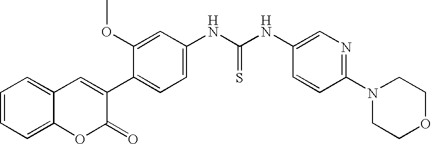

### Compound 08: 1-[3-methoxy-4-(2-oxo-2H-chromen-3-yl)-phenyl]-3-(6-morpholin-4-yl-pyridin-3-yl)-thiourea

Yellow solid.

NMR (250 MHz, DMSO): δ 9.87 (s, 1H), 9.62 (s, 1H), 8.10 (d, J 2.7, 1H), 8.01 (s, 1H), 7.75 (dd, J 8.2 and 0.7, 1H), 7.70-7.55 (m, 2H), 7.45-7.25 (m, 4H), 7.12 (dd, J 8.0 and 1.5, 1H), 6.83 (d, J 8.7, 1H), 3.80-3.65 (m, 7H), 3.45-3.35 (m, 4H).

LC-MS purity: 92%

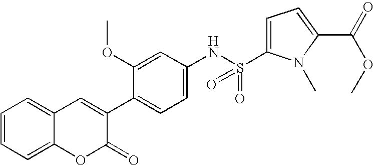

### Compound 09: methyl 5-({[3-methoxy-4-(2-oxo-2H-chromen-3-yl)phenyl]amino}sulfonyl)-1-methyl-1H-pyrrole-2-carboxylate: (Alternative Name: 5-[3-methoxy-4-(2-oxo-2H-chromen-3-yl)-phenylsulfamoyl]-1-methyl-1H-pyrrole-2-carboxylic acid methyl ester)

Yellow solid

NMR (250 MHz, DMSO): δ 10.30 (br s, 1H), 7.96 (s, 1H), 7.78 (d, J 2.0, 1H), 7.70 (dd, J 8.0 and 1.5, 1H), 7.60 (td, J 8.2 and 1.5, 1H), 7.45-7.30 (m, 2H), 7.21 (d, J 8.2, 1H), 7.03 (d, J 2.0, 1H), 6.86 (d, J 2.0, 1H), 6.77 (dd, J 8.2 and 2.0, 1H), 3.86 (s, 3H), 3.75 (s, 3H), 3.68 (s, 3H).

LC-MS purity: 96%

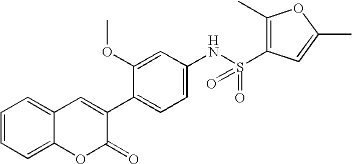

### Compound 13: N-[3-methoxy-4-(2-oxo-2H-chromen-3-yl)phenyl]-2,5-dimethylfuran-3-sulfonamide. (Alternative Name: 2,5-dimethyl-furan-3-sulfonic acid [3-methoxy-4-(2-oxo-2H-chromen-3-yl)-phenyl]-amide)

Off-white solid.

NMR (250 MHz, DMSO): δ 10.41 (s, 1H), 7.98 (s, 1H), 7.71 (d, J 7.5, 1H), 7.61 (t, J 8.0, 1H), 7.43-7.33 (m, 2H), 7.23 (d, J 8.0, 1H), 6.84 (s, 1H), 6.75 (d, J 8.2, 1H), 6.26 (s, 1H), 3.68 (s, 3H), 2.41 (s, 3H), 2.21 (s, 3H).

LC-MS purity: 97%

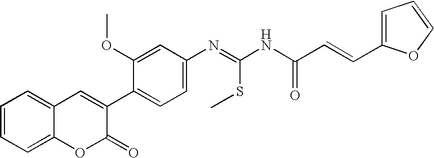

### Compound 14: methyl N-[(2E)-3-(2-furyl)prop-2-enoyl]-Ar-[3-methoxy-4-(2-oxo-2H-chromen-3-yl)phenyl]imidothiocarbamate (Alternative Name: 1-((E)-3-furan-2-yl-acryloyl)-3-[3-methoxy-4-(2-oxo-2H-chromen-3-yl)-phenyl]-2-methyl-isothiourea)

Yellow solid.

NMR (250 MHz, CDCl₃): δ 7.79 (s, 1H), 7.70-7.25 (m, 7H), 7.05-6.85 (m, 2H), 6.75-6.45 (m, 3H), 3.83 (s, 3H), 2.57 (br s, 3H).

LC-MS purity: 96%

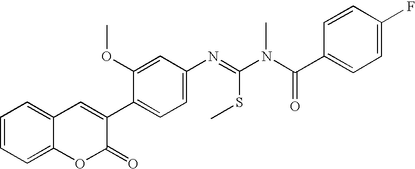

### Compound 25: 1-(4-fluoro-benzoyl)-3-[3-methoxy-4-(2-oxo-2H-chromen-3-yl)-phenyl]-1,2-dimethyl-isothiourea

White solid.

NMR (250 MHz, DMSO): δ 8.20-8.05 (m, 3H), 7.85-7.60 (m, 2H), 7.55-7.20 (m, 6H), 7.15-7.05 (m, 1H), 3.78 (s, 3H), 3.45 (s, 3H), 2.19 (s, 3H).

LC-MS purity: 97%

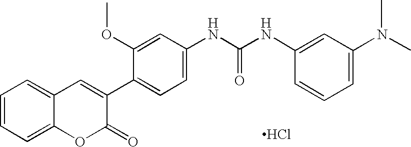

### Compound 18: 1-(3-dimethylamino-phenyl)-3-[3-methoxy-4-(2-oxo-2H-chromen-3-yl)-phenyl]-urea.HCl

Off-white solid.

NMR (250 MHz, DMSO): δ 8.85 (br s, 1H), 8.65 (br s, 1H), 8.00 (s, 1H), 7.74 (dd, J 7.5 and 0.75, 1H), 7.61 (td, J 7.9 and 1.5, 1H), 7.45-6.95 (m, 7H), 6.90-6.75 (m, 1H), 6.50-6.35 (m, 1H), 3.75 (s, 3H), 2.91 (s, 6H).

LC-MS purity: 99%

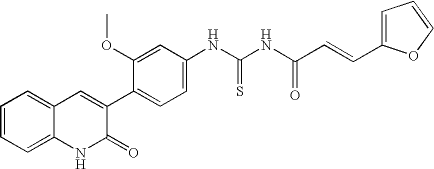

### Compound 15: (2E)-3-(2-furyl)-N-({[3-methoxy-4-(2-oxo-1,2-dihydroquinolin-3-yl)phenyl]amino}carbonothioyl)acrylamide (Alternative Name: 1-((E)-3-furan-2-yl-acryloyl)-3-[3-methoxy-4-(2-oxo-1,2-dihydro-quinolin-3-yl)-phenyl]-thiourea)

Yellow solid.

NMR (250 MHz, DMSO): δ 12.87 (s, 1H), 11.84 (s, 1H), 11.70 (s, 1H), 8.00-7.85 (m, 2H), 7.75-7.22 (m, 7H), 7.18 (t, J 7.5, 1H), 7.01 (d, J 3.2, 1H), 6.81 (d, J 15.5, 1H), 6.70-6.66 (m, 1H), 3.74 (s, 3H).

LC-MS purity: 92%

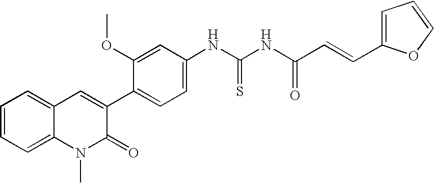

### Compound 17: (2E)-3-(2-furyl)-N-({[3-methoxy-4-(1-methyl-2-oxo-1,2-dihydroquinolin-3-yl)phenyl]amino}carbonothioyl)acrylamide

### Alternative Name: 1-((E)-3-furan-2-yl-acryloyl)-3-[3-methoxy-4-(1-methyl-2-oxo-1,2-dihydro-quinolin-3-yl)-phenyl]-thiourea

Yellow solid.

NMR (250 MHz, DMSO): δ 12.88 (br s, 1H), 11.71 (br s, 1H), 7.95-7.85 (m, 2H), 7.74 (d, J 7.7, 1H), 7.70-7.50 (m, 4H), 7.28-7.20 (m, 3H), 7.01 (d, J 3.5, 1H), 6.82 (d, J 15.5, 1H), 6.70-6.68 (m, 1H), 3.73 (s, 3H), 3.68 (s, 3H).

LC-MS purity: 93%

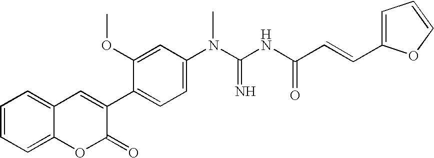

### Compound 19: N′-((E)-3-furan-2-yl-acryloyl)-N-[3-methoxy-4-(2-oxo-2H-chromen-3-yl)-phenyl]-N-methyl-guanidine

Off-white solid.

NMR (250 MHz, CDCl₃): δ 8.06 (br s, 1H), 7.85-6.95 (m, 9H), 6.80-6.75 (m, 1H), 6.60-6.55 (m, 1H), 6.31 (d, J 15.5, 1H), 3.79 (br s, 3H), 3.56 (brs, 3H).

LC-MS purity: 99%

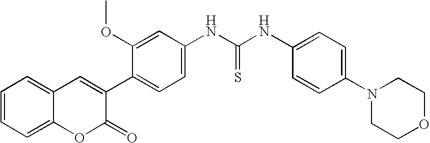

### Compound 20

### 1-[3-methoxy-4-(2-oxo-2H-chromen-3-yl)-phenyl]-3-(4-morpholin-4-yl-phenyl)-thiourea

Yellow solid.

NMR (250 MHz, DMSO): δ 9.70 (br s, 2H), 7.99 (s, 1H), 7.73 (d, J 7.7, 1H), 7.61 (t, J 7.2, 1H), 7.45-7.20 (m, 6H), 7.10 (dd, J 8.5 and 1.7, 1H), 6.91 (d, J 8.7, 2H), 3.75-3.65 (m, 7H), 3.10-3.3.05 (m, 4H).

LC-MS purity: 93%

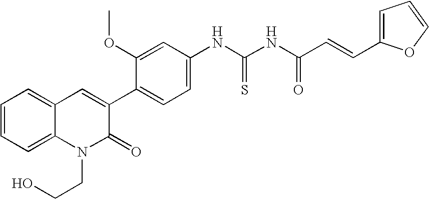

### Compound 21

### 1-((E)-3-furan-2-yl-acryloyl)-3-{4-[1-(2-hydroxy-ethyl)-2-oxo-1,2-dihydro-quinolin-3-yl]-3-methoxy-phenyl}-thiourea

Pale yellow solid.

NMR (250 MHz, DMSO): δ 12.87 (br s, 1H), 11.70 (br s, 1H), 7.95-7.85 (m, 2H), 7.90-7.55 (m, 5H), 7.20-7.10 (m, 3H), 7.01 (d, J 3.5, 1H), 6.82 (d, J 15.7, 1H), 6.70-6.60 (m, 1H), 4.95 (t, J 5.2, 1H), 4.37 (t, J 6.5, 2H), 3.80-3.60 (m, 5H).

LC-MS purity: 97%

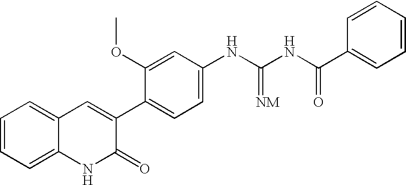

### Compound 22: N-Benzoyl-N′-[3-methoxy-4-(2-oxo-1,2-dihydro-quinolin-3-yl)-phenyl]-guanidine

White solid.

NMR (250 MHz, DMSO): δ 11.82 (s, 1H), 9.40 (br s, 1H), 8.17-8.12 (m, 2H), 7.87 (s, 1H), 7.66 (d, J 8.0, 1H), 7.60-7.25 (m, 8H), 7.20 (t, J 7.5, 1H), 7.02 (d, J 6.7, 1H), 3.77 (s, 3H).

LC-MS purity: 93%

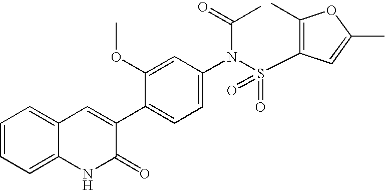

### Compound 23: 2,5-dimethyl-furan-3-sulfonic acid acetyl-[3-methoxy-4-(2-oxo-2H-chromen-3-yl)-phenyl]-amide

White foam.

NMR (250 MHz, CDCl₃): δ 7.83 (s, 1H), 7.65-7.45 (m, 3H), 7.45-7.20 (m, 2H), 6.95-6.70 (m, 2H), 6.26 (s, 1H), 3.85 (s, 3H), 2.57 (s, 3H), 2.29 (s, 3H), 2.01 (s, 3H).

LC-MS purity: 86%

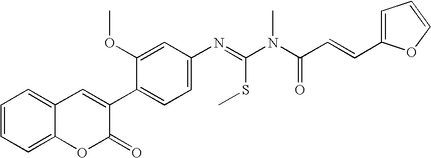

### Compound 24: 1-((E)-3-furan-2-yl-acryloyl)-3-[3-methoxy-4-(2-oxo-2H-chromen-3-yl)-phenyl]-1,2-dimethyl-isothiourea

Yellow foam.

NMR (250 MHz, DMSO): δ 8.02 (br s, 1H), 7.85 (br s, 1H), 7.72 (d, J 8.2, 1H), 7.61 (t, J 7.5, 1H), 7.45-7.20 (m, 4H), 6.92 (br s, 1H), 6.70-6.35 (m, 4H), 3.55 (br s, 3H), 3.05 (br s, 3H), 2.55 (br s, 3H).

LC-MS purity: 98%

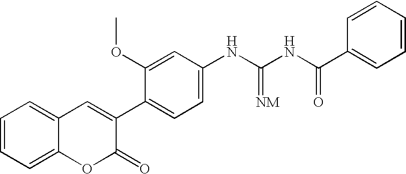

### Compound 07: N-(imino{[3-methoxy-4-(2-oxo-2H-chromen-3-yl)phenyl]amino}methyl)benzamide

### Alternative Name: N-Benzoyl-N′-[3-methoxy-4-(2-oxo-2H-chromen-3-yl)-phenyl]-guanidine

White solid.

NMR (250 MHz, CDCl₃): δ 8.17-8.12 (m, 2H), 7.74 (s, 1H), 7.60-7.20 (m, 8H), 6.85-6.75 (m, 2H), 3.73 (s, 3H).

LC-MS purity: 100%

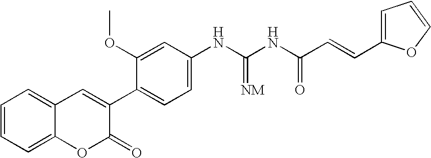

### Compound 12: (2E)-3-(2-furyl)-N-(imino{[3-methoxy-4-(2-oxo-2H-chromen-3-yl)phenyl]amino}methyl)acrylamide

### Alternative Name: N-((E)-3-furan-2-yl-acryloyl)-N′-[3-methoxy-4-(2-oxo-2H-chromen-3-yl)-phenyl]-guanidine

White solid.

NMR (250 MHz, DMSO): δ 11.82 (br s, 1H), 10.93 (br s, 1H), 9.13 (br s, 1H), 8.03 (s, 1H), 7.96 (s, 1H), 7.79 (d, J 7.7, 1H), 7.70-7.60 (m, 2H), 7.55-7.35 (m, 3H), 7.19 (d, J 1.0, 1H), 7.15-7.00 (m, 2H), 6.80-6.60 (m, 2H), 3.79 (br s, 3H).

LC-MS purity: 95%

### Example 2

### Test for Proliferation/Survival In Vitro and Determination of Cell Cycle

### 2A Propidium Iodide Staining (Assay for Cytotoxic Effect)

Propidium iodide (PI) cannot normally cross the cell membrane; it is thus excluded by viable cells but not from dying or dead cells, having a damaged membrane, where it intercalates into double-stranded nucleic acids and fluoresces when excited at 488 nm.

In order to determine the cytotoxic effect of a compound, 2.10⁵ cells are incubated with the compound to be tested during a time ranging from hours to days, then the cells are collected either by simple pipeting if the cells grow in suspension or after mild trypsinization for adherent cells. The cells are pelleted by gentle centrifugation 1200 rpm/5 min/4° C., then the supernatant is discarded and replaced by 1 mL of fresh medium. One hundred microliters of propidium iodide (40 μg/mL) are added to 400 μL of the cell suspension in a FACS tube and viability is measured by flow cytometry as the percentage of cells whose fluorescence recorded on FL-3 channel is comprised between 0 and 2.

### 2B. Cell Cycle Analysis

In order to determine the cytostatic effect of a compound, 2.10⁵ cells are incubated with the compound to be tested during a time ranging from hours to days, then the cells are collected either by simple pipeting if the cells grow in suspension or after mild trypsinization for adherent cells. The cells are pelleted by gentle centrifugation 1200 rpm/5 min/4° C., then the supernatant is discarded and the cells are fixed with 1 mL of PBS/Ethanol 70% overnight at −20° C. The tubes are then centrifuged during 5 minutes at 1200 rpm and the supernatant is removed. The pellet is then re-suspended in 450 μL of PBS before 50 μL of RNAse (1 mg/mL) and 5 μL of propidium iodide (2 mg/mL) are added. Each tube is left 30 minutes at 37° C. and immediately analysed by flow cytometry after incubation.

A biparametric dot plot representation FL3 peak vs FL3 integer is plotted and the cells located on the diagonal are gated and analysed on a histogram representing the number of cells as a function of the FL3 peak.

To assess the relative position of cursors defining relative proportion of cells in subG1, G1, S or G2 phase of the cell cycle, G1 and G2 peaks are to be defined first:

The G1 peak is defined as the major peak in absence of treatment (generally X=200);

The G2 peak is centered on the value which is twice the value of the G1 peak (generally X=400).

Cells in subG1 phase are plotted in the part of the histogram preceding the G1 peak (generally X<200) and cells in S phase are enclosed in the area between the G1 and G2 peaks.

On this graphic, subG1, G1, S and G2/M phases of the cell cycle can thus be defined.

2C Proliferation test ViaLight® HS High Sensitivity Cytotoxicity and Cell Proliferation BioAssay Kit

This protocol allows the determination of the cytostatic and cytotoxic activities of compounds to be tested on mammalian cells in culture and was carried out essentially according to the manufacturer's recommendations.

Cells are suspended in 15 mL of their respective media and are enumerated. They are then diluted in their media in order to obtain:
2500 cells per 90 μL per well for MRC5 cells
5000 cells per 90 μL per well for all other cell lines.
   Cells are then seeded in a 96 well plate, and incubated at least 4 hours in the incubator at 37° C. in 5% CO₂.

The molecules to be tested are diluted in DMEM with DMSO in order to obtain the following dilutions:
dilution at 250 μM/1% DMSO
dilution at 50 μM/1% DMSO
dilution at 1 μM/1% DMSO
dilution at 2 μM/1% DMSO
dilution at 400 nM/1% DMSO
dilution at 80 nM/1% DMSO
   For each well comprising 90 μL of cell suspension, 10 μL of a solution of the compound to be tested is added, with the 6 different concentrations. The plate is then incubated during 72 hours. One control plate is contacted with 10 μL of DMEM 1% DMSO, without compound to be tested. 50 μL of lysis buffer (test Vialight® lysis buffer) is added in each well of the control plate and the plate is incubated 10 minutes under hood.

Then, 100 μL of test buffer (test Vialight® test buffer) is added in each well and the control plate is incubated during 2 minutes. The luminescence is read in each well. The mean luminescence for each type of cells is called T0.

After the 72 h incubation, in order to determine the viability, 50 μL of lysis buffer (test Vialight® lysis buffer) is added in each well of the plates and the plates are incubated 10 minutes under hood.

Then, 100 μL of test buffer (test Vialight® test buffer) is added in each well and the plates are incubated during 2 minutes. The luminescence is read in each well. For each type of cell, the mean luminescence in the well not containing the compound is called C72 and the mean luminescence in well containing a given concentration of a compound to be tested is called T72. For each dilution (concentration of compound to be tested), T0 and T72 values are compared. If T72 is superior or equal to T0, the following formula is applied to determine the growth rate:

Y=Growth (%)=(T72−T0)/(C72−T0)×100

If T72 is inferior to T0, then the following formula is applied to determine the lethality rate:

Y=LC(%)=(T72−T0)/T0×100

The graph is plotted for the different concentrations of compound and
GI50 (50% of growth inhibitory) is determined, this is the dose producing 50% of cell growth (intersection at Y=50);
TGI (total growth inhibitory) is determined on the graph, this is the dose producing the total arrest of the cell growth (intersection at Y=0); and
LC50 (50% of lethal concentration) is determined, this is the dose producing the death of 50% of the cells (intersection at Y=−50).

### Example 3

### Inhibition of Cell Proliferation In Vitro with Compound (01)

The effect of a compound of the invention, compound (01): (2E)-3-(2-furyl)-N-({[3-methoxy-4-(2-oxo-2H-chromen-3-yl)phenyl]amino}carbonothioyl)acrylamide, on cell proliferation has been tested on different cells, i.e. MRC5 (normal non-transformed lung fibroblasts), H146 (small cell lung carcinoma), H69 (small cell lung carcinoma), H69AR (small cell lung carcinoma, adriamycin resistant), U937 (histiocytic lymphoma), BL41 (Burkitt Lymphoma), Val (lymphoma), RS4-11 (acute lymphoblastic leukaemia), H460 (non-small cell lung carcinoma), PC3 (prostate adenocarcinoma), MDA231 (breast epithelial adenocarcinoma), MCF7 (breast epithelial adenocarcinoma), Lovo (colorectal adenocarcinoma) and A375 (melanoma), at different concentrations. The protocol is detailed in example 2C.

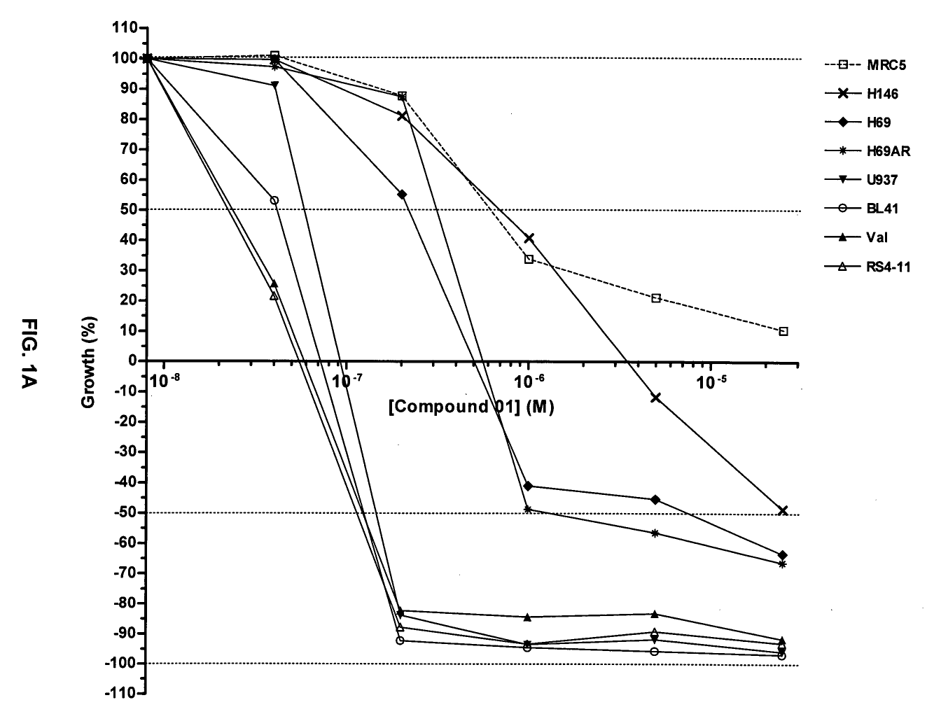

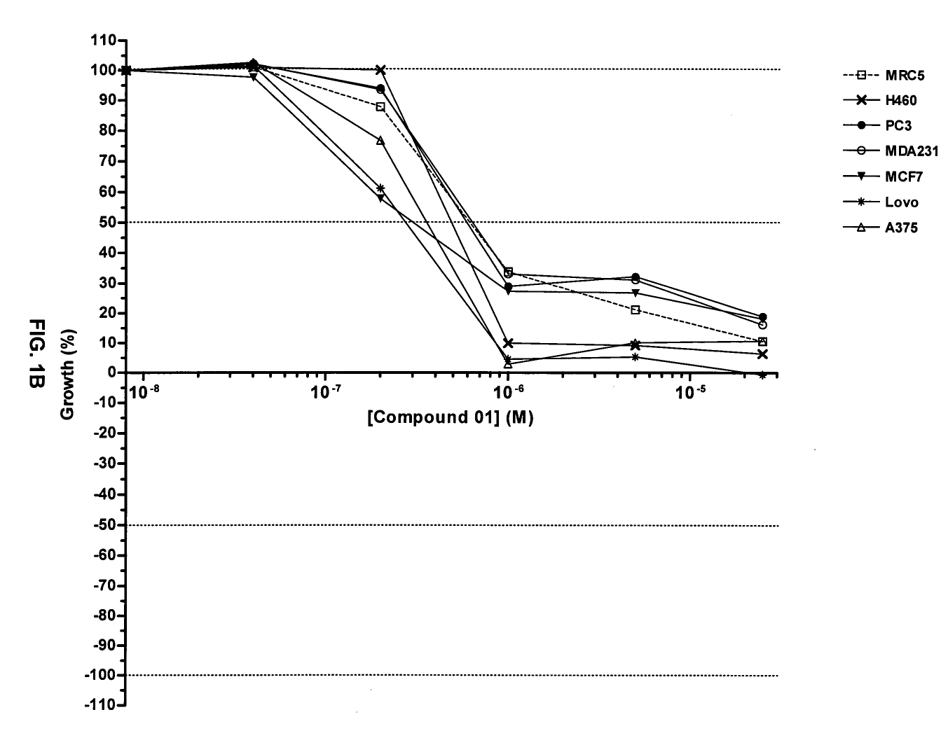

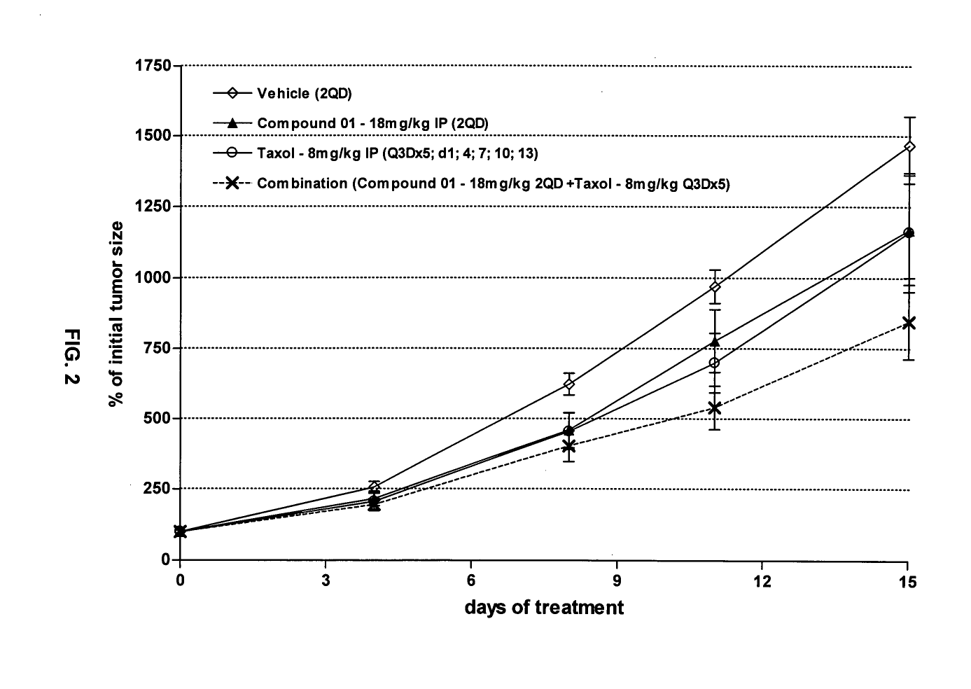

The results are depicted in FIGS. 1A and 1B.

A negative percentage of cell growth implies a cytotoxic effect, in addition to an antistatic effect. In conclusion, it can be seen that compound (01) inhibits the growth of lung, prostate, breast, colon, melanoma, and myeloid tumor cells with IC₅₀s in the range of 50 nM to 1 μM.

Viability and cell cycle profile, tested with compound (01), at 500 nM:

| | | Viabil- | Viabil- | Cell cycle profile - |
|---|---|---|---|---|
| Cell line | Origin | ity 48 h | ity 72 h | 72 h |
| MRC5 | Non-transformed | 94% | 89% | G2/M accumulation |
| | fibroblasts | | | |
| PC3 | Prostate | 92% | 90% | G2/M accumulation |
| NCI-H460 | Lung (NSCLC) | 80% | 80% | G2/M accumulation |
| | | | | Strong sub-G1 |
| A375 | Melanoma | 65% | 35% | G2/M accumulation |
| | | | | Strong sub-G1 |
| U937 | Myeloid | 25% | 6% | Strong sub-G1 |
| | | | | accumulation |
| CEM | Lymphoid | 10% | 2% | Strong sub-G1 |
| Jurkat | — | 4% | 1% | accumulation |

In conclusion, it can be seen that the compound (01) ((2E)-3-(2-furyl)-N-({[3-methoxy-4-(2-oxo-2H-chromen-3-yl)phenyl]amino}carbonothioyl)acrylamide) has cytotoxic effect on lymphoid and myeloid tumor cells, with an apoptotic cell cycle profile and has cytostatic effect on prostate and lung tumor cells by cell cycle arrest at entry into mitosis.

It is also observed that control non-transformed cells (MRC5) are unaffected by contact with the molecule (01) in this experiment.

### Example 4

### Inhibition of Tumor Growth In Vivo

For this experiment, Swiss nude mice received a subcutaneous xenograft of 5×10⁶ human lung cancer NCI-H460 cells (NSCLC). The percentage of growth of the tumor size versus tumor size at the beginning of treatment is plotted during 15 days for mice receiving four different treatments (12 mice per group of treatment).

The first treatment corresponds to a control. The mice were given the vehicule intra-peritoneally, 2QD.

Composition of the vehicule: 10% DMSO; 10% PEG-400; 30% (EtOH/Cremophor (1/2)); 50% sterile water.

The second treatment corresponds to the administration of a compound of the invention, compound (01), at a dosage of 18 mg/kg 2QD.

The third treatment corresponds to the administration of Taxol at a dosage of 8 mg/kg Q3D×5; d1; d4; d7; d10; d13.

The fourth treatment corresponds to the administration of a combination of compound (01) at a dosage of 18 mg/kg 2QD and of Taxol at a dosage of 8 mg/kg Q3D×5.

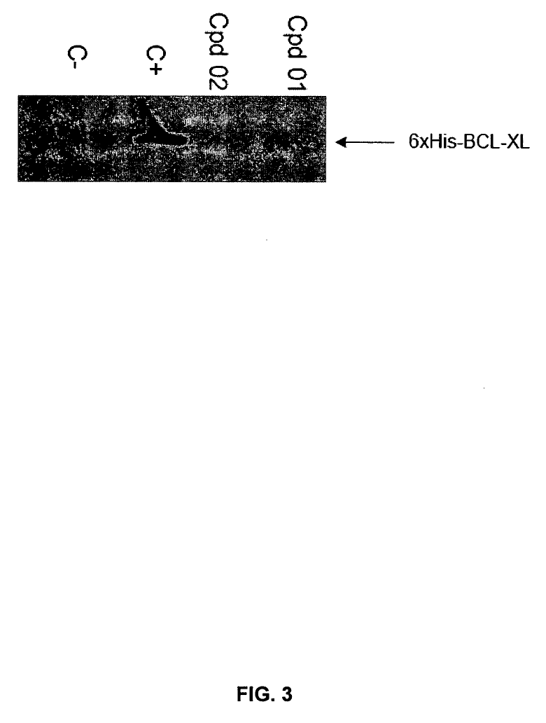

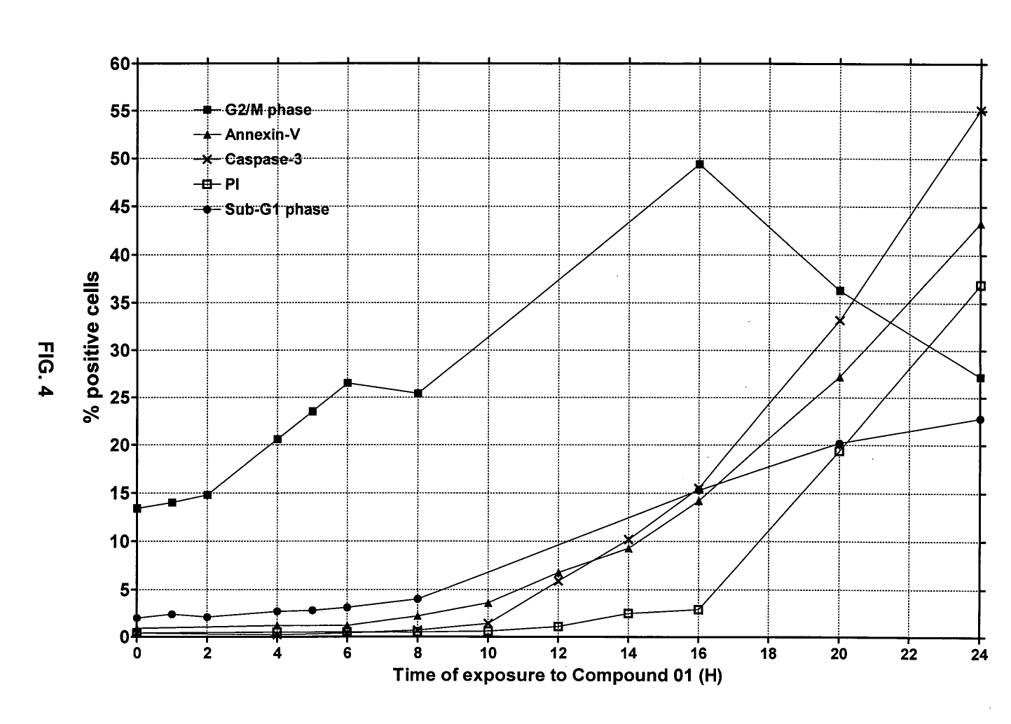

The results are depicted in FIG. 2.

It can be concluded that the compound (01) ((2E)-3-(2-furyl)-N-({[3-methoxy-4-(2-oxo-2H-chromen-3-yl)phenyl]amino}carbonothioyl)acrylamide) inhibits the growth of an aggressive lung tumor xenograft model. It is also observed that the efficacy of compound (01) in reducing the tumour growth is enhanced when combined with another anti-tumour drug, especially when combined with taxol. It has also been observed that the inhibition occurs in a dose-dependent fashion.

### Example 5

Direct binding of compound (01) on Bcl-xL determined by fluorescence interference

Experiments were performed by using a SLM-Aminco 8000C spectrofluorometer with spectral bandwidths of 2 and 4 nm, respectively, for excitation and emission.

0.5 μM purified 6×His-Bcl-xL were diluted in 1.2 mL of the following buffer pre equilibrated at 25° C. (10 mM Tris-HCl, pH=7.4; 100 mM NaCl; 1 mM β-mercapto ethanol and 10% glycerol). Fluorescent measurements were performed with increasing concentrations of molecule (01) (from 100 nM to 7.5 μM) in 100% DMSO

Tryptophan-intrinsic fluorescence of Bcl-xL was measured by scanning emission in the range 310-390 nm upon excitation at 295 nm. The binding of compound (01) was monitored by the increase of fluorescence with increasing concentrations of compound (01).

Correction for innerfilter effect and DMSO dilution were determined using NATA (N-acetyltryptophanamide). All spectra were corrected for the effects of buffer and for dilution. Curve fitting was performed using Grafit (Erithacus software), with the following quadratic equation for increasing fluorescence:

F=Fmin+{(Fmax−Fmin)[(Et+L+Kd)−((Et+L+Kd)2−4EtL)1/2]}/2Et

Where: F=relative fluorescence intensity
Fmin=Fluorescence intensity at the start of titration
Fmax=Fluorescence at saturating concentration of compound (01) (Ligand=L)
Et=Total concentration of 6×His-Bcl-xL
Kd=Apparent dissociation constant of 6×His-Bcl-xU compound (01) complex.

### Example 6

### Pull Down Assay with Small Molecules

A peptide aptamer (dubbed BF8) has been selected against the anti-apoptotic protein Bfl1, member of the BCL-2 family. BF8 was shown to interact also with other members of the BCL-2 family, among which Bcl-xL. Two lines of evidence indicate that BF8 binds the hydrophobic groove of anti-apoptotic BCL-2-like proteins. First, the amino-acid sequence of its variable region bears similarities with the sequence of the BH3 domains of pro-apoptotic members of the BCL-2 family (such as Bak or Bax), known to bind the anti-apoptotic proteins via this hydrophobic groove. Second, BF8 no longer binds a mutated variant of Bfl-1 that bears an amino-acid substitution within the said hydrophobic groove.

A Bcl-xL/BF8 in vitro interaction assay was developed to examine the capacity of the compounds of the invention to disrupt this interaction by competition with the peptide aptamer. 6×His-Bcl-xL and GST-BF8 recombinant fusion proteins were expressed in E. coli and purified using affinity chromatography. The GST-BF8 fusion protein (or a GST-control aptamer fusion protein) were coupled to glutathione-sepharose solid phases, and soluble 6×His-Bcl-xL was added to these phases, in presence of different molecules. 6×His-Bcl-xL molecules captured by the solid phases were revealed by a Western Blot experiment using an anti-6×His antibody.

Details of the protocol are the following:
1—25 μL glutathion-Sepharose beads pre-rinsed are mixed with pull down buffer (50 mM Tris-HCl, pH=8.0; 100 mM NaCl; 1 mM DTT et 0.01% Tween20) with 10 μG purified GST-BF8 fusion protein. The mixture is incubated at room temperature for half an hour.
2—Beads are washed by centrifugation and dilution in 1 mL pull down buffer. Beads are then collected by centrifugation and the supernatant is removed.
3—0.5 μG of purified 6×His-Bcl-xL fusion protein, pre incubated at 4° C. for two hours with molecules (50 μM) in pull down buffer supplemented with 5% DMSO, is added. The mixture is incubated for two hours under constant agitation.
4—The beads are then washed with three serial concentration/dilution in 1 mL of pull down buffer.
5—The beads are recovered, supernatant is removed and the beads are resuspend in 1× Laemli buffer for SDS-PAGE (0.25 M Tris-HCl, pH=6.8; 5% SDS; 0.025% bromophenol blue; 0.6 M β-mercaptoethanol and 10% glycerol). The mixture is heated for 10 min at 100° C. before SDS-PAGE.
6—Western blot analysis is performed using an anti-His tag antibody

From the result shown in FIG. 3, it can be concluded that compounds (01) ((2E)-3-(2-furyl)-N-({[3-methoxy-4-(2-oxo-2H-chromen-3-yl)phenyl]amino}carbonothioyl)acrylamide) and (02) (N-({[3-methoxy-4-(2-oxo-2H-chromen-3-yl)phenyl]amino}carbonothioyl)benzamide) inhibit the in vitro interaction between 6×His-Bcl-xL and GST-BF8.

### Example 7

### Apoptosis

In order to demonstrate that the cytotoxic activity of the compounds of this invention is caused by the induction of apoptosis (as can be expected from the targeting of BCL-2-like proteins), three different assays that specifically detect apoptosis were performed on U937 cells treated with compound (01), over a time-course of 24 h. The percentage of cells showing activated Caspase 3 and phosphadityl serine exposure at the outer face of the plasma membrane were determined by flow cytometry, according the enclosed protocols. Both assays enable to detect early markers of apoptosis. The percentage of cells exhibiting a sub-G1 phase DNA content was also determined by flow cytometry. Such DNA content is classically used as a marker of apoptosis. In the experiment shown in FIG. 4, the percentage of cells exhibiting a G2/M phase DNA content was also determined. Finally, in this same experiment, the percentage of dead cells was determined by incorporation of propidium iodine.

Details of the protocol are the following:

### Cell Culture and Compound (01) Treatment

U937 human histiocytic lymphoma were obtained from European Collection of Cell Cultures (ECACC 85011440) and maintained in suspension in RPMI 1640 medium supplemented with 10% heat decomplemented fetal bovine serum, 100 Ul/ml penicillin, 100 μg/ml streptomycin and 2 mM glutamax I.

Twelve hours before addition of compound (01), U937 cells were seeded in 12 well plates at density of 5×10⁵ cells/900 μl of medium.

Cells were treated by 100 μl of 5 μM compound (01)/1% DMSO (final concentration 0.5 μM/0.1% DMSO) for various time of drug exposure, ranging from 4 hours to 24 hours or DMSO alone for 10 and 24 hours (Control 1, Control 2 respectively)

Wells corresponding to 4, 6, 8, 10 hours of incubation and Control 1 were analyzed on day 1.

Wells corresponding to 12, 14, 16, 20, 24 hours of incubation and Control 2 were analyzed on day 2.

Annexin-V binding analysis and Propidium Iodide staining (PI) were performed by flow cytometry with Annexin-V FITC kit (IM3546.; Beckman Coulter; Villepinte) according to manufacturer instructions.

Briefly, 5×10⁵ cells were pelleted by centrifugation 1600 rpm/4° C., resuspended in 100 μl of ice-cold 1× Annexin-V Binding Buffer containing 1 μl of Annexin V-FITC solution and 5 μl of PI (250 μg/ml). After 15 minutes of incubation on ice in the dark, 400 μl of 1× binding buffer were added and cell preparations analysed by flow cytometry.

Caspase-3,7 activity analysis was performed with by flow cytometry with Caspglow™ kit (K-183/biovision; Mountain View) according to manufacturer instruction.

Briefly, 5×10⁵ cells were pelleted by centrifugation 1600 rpm/4° C. and resuspended in 300 μl cell culture medium. One microliter of FITC-DEVD-FMK was added into each tube and left for 30 minutes at 37° C. in incubator with 5% CO₂. Cells were centrifuged twice (3000 rpm/5 minutes) and washed with 0.5 ml of Wash Buffer before being resuspended in 300 μl of Wash buffer and kept on ice until analysis by flow cytometry.

### Flow Cytometry.

All experiments were carried out on a Cytomics Flow Cytometer 500 (Beckman Coulter) equipped with air-cooled Argon ion laser 488 nm. All analysis were performed using CXP software (Beckman Coulter).

For each data point, a minimum of 20.000 cells were analysed.

Caspase-3,7 activity and Annexin-V binding were quantified using the FL-1 channel while PI staining was quantified using the FL-4 channel.

Cell cycle analysis was performed as previously described.

From the result shown here and in FIG. 4, it can be concluded that compound (01) (((2E)-3-(2-furyl)-N-({[3-methoxy-4-(2-oxo-2H-chromen-3-yl)phenyl]amino}carbonothioyl)acrylamide)) exerts a cytostatic effect on U937 cells (as revealed by an early increase of the cell population in the G2/M phase of the cell cycle) and induces apoptosis (as revealed by the gradual increase of the percentage of cells exhibiting activated caspase 3, membrane exposure of phosphatidylserine, sub-G1 DNA content).

### Example 8

### Proliferation Test

Following the protocol described in Example 2C, different compounds according to the invention have been tested for their cytostatic and cytotoxic activities on mammalian cells in culture (specifically U937 cells).

The GI50 (50% of growth inhibitory) has been determined for all the following compounds, i.e. the dose producing 50% of cell growth inhibition.

The results obtained are given in the following table. Where the GI50 provided is in the form of a range, it means that multiple experiments, whose results fall within the range indicated, were performed. Otherwise, results of individual experiments are provided.

| Compound | Structure | U937 GI50 (μM) |
|---|---|---|
| Compound 01 | 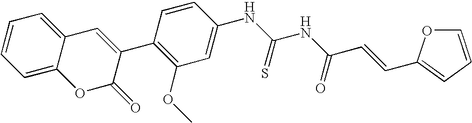 | 0.059-0.2 |
| Compound 14 | 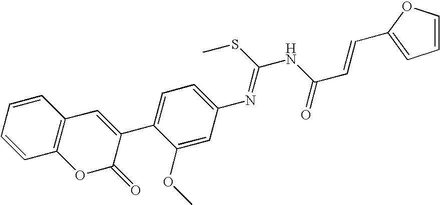 | 0.22 |
| Compound 24 | 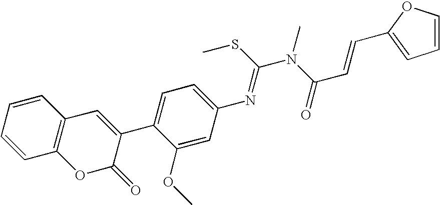 | 0.033 |
| Compound 02 | 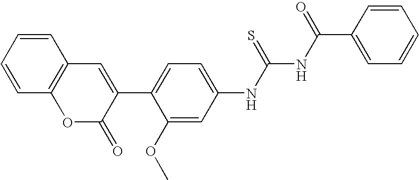 | 0.6-0.86 |
| Compound 05 | 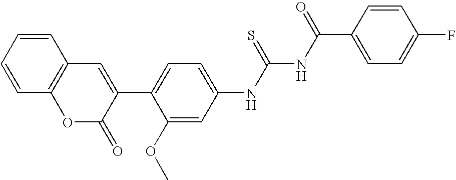 | 0.92, 1.5 |
| Compound 25 | 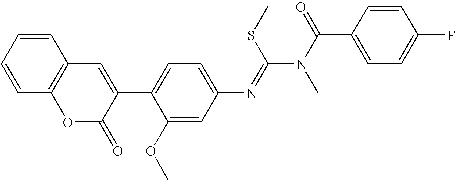 | 0.41 |
| Compound 07 | 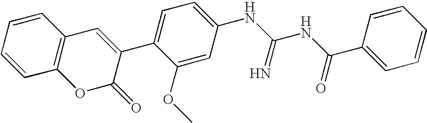 | 0.63, 0.8 |
| Compound 12 | 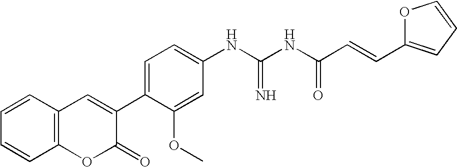 | 1.6-2.1 |
| Compound 19 | 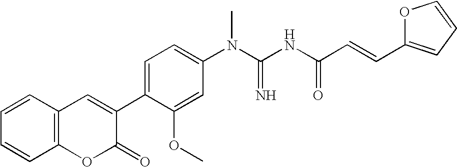 | 9.2 |
| Compound 03 | 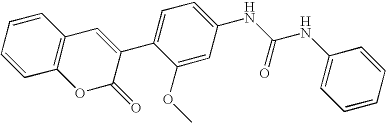 | 0.61 |
| Compound 06 | 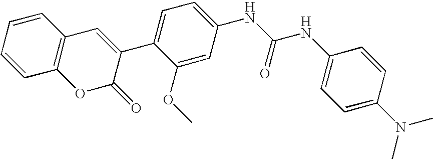 | 0.32, 0.58 |
| Compound 18 | 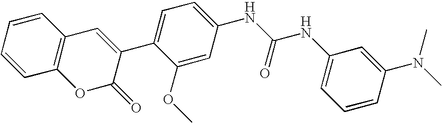 | 0.15 |
| Compound 08 | 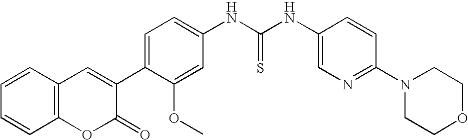 | 1.2 |
| Compound 20 | 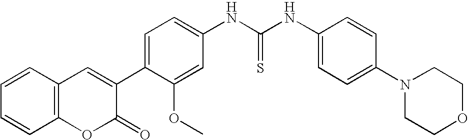 | 1.1 |
| Compound 15 | 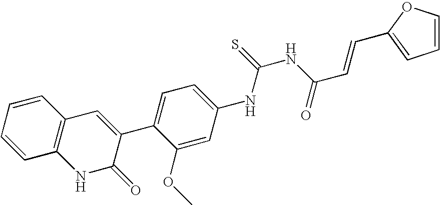 | 0.063, 0.21 |
| Compound 17 | 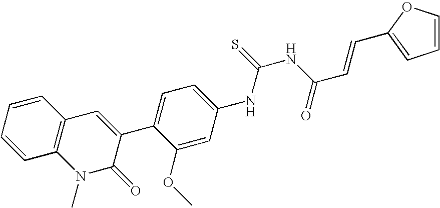 | 0.045 |
| Compound 21 | 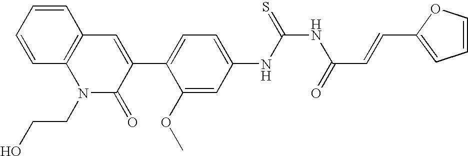 | ≈0.09 |
| Compound 22 | 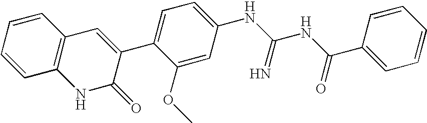 | 1 |
| Compound 09 | 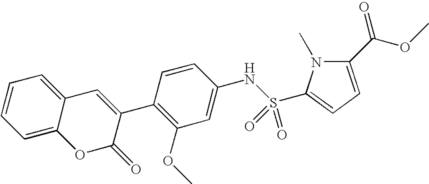 | 0.5-0.6 |
| Compound 13 | 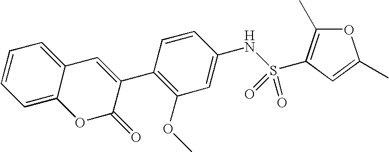 | 0.1 |
| Compound 23 | 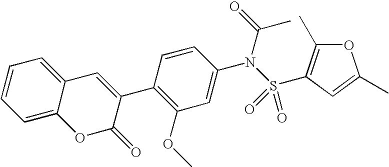 | ≈0.5 |

### BIBLIOGRAPHY

Contractor R, Konopleva M, Samudio I, et al. Inhibition of Bcl-2 signaling by small molecule BH3 inhibitor GX15-070 as a novel therapeutic strategy in AML. Blood. 2005; 106:942a, abstract # 3372.
Galan P P, Roue G, Billamor N, et al. The small molecule pan=Bcl-2 inhibitor GX15-070 induces apoptosis in vitro in mantle cell lymphoma (MCL) cells and exhibits a synergistic effect in combination with the proteasome inhibitor bortezomib (Velcade®). Blood. 2005; 106:429a, abstract #1490.
Mohammad R M, Wang S, Aboukameel A, Chen B, Wu X, Chen J, Al-Katib A. Preclinical studies of a nonpeptidic small-molecule inhibitor of Bcl-2 and Bcl-X(L) [(−)-gossypol] against diffuse large cell lymphoma. Mol Cancer Ther. 2005 January; 4(1):13-21.
Oltersdorf T. et al. An inhibitor of Bcl-2 family proteins induces regression of solid tumours. Nature. 2005; 435 (7042): 677-81.
Sattler M. et al. Structure of Bcl-xL-Bak peptide complex: recognition between regulators of apoptosis. Science. 1997; 275 (5302): 983-986.

1. Compound with anti-proliferative properties having one of the general formula: 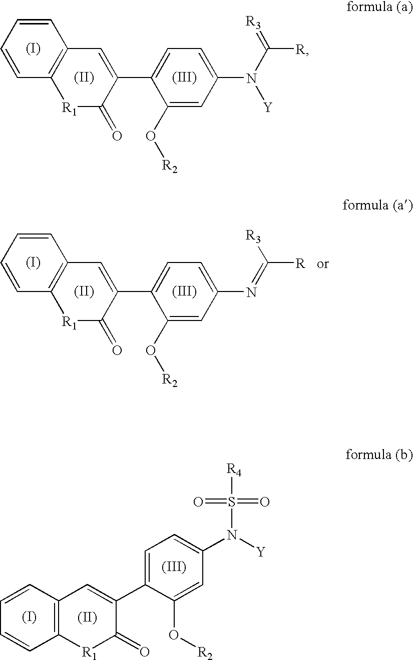
wherein the cycles (I), (II) and (III) may be optionally substituted on 1, 2, 3 or 4 of the available carbons with a group chosen amongst: Br₁ Cl, F, I, OH₁0-CH₃, COOH, and (CH₂)ₘ—R₉, wherein R₉ represents H, CH₃, OH, O—CH₃, COOH₁ NH₃, CO—NH₂, NH—CO-phenyl, an alkyl, an heteroaryl or an heterocycloalkyl and m=1, 2, 3 or 4.
wherein Y represents H, CH₃ or CO—CH₃;
wherein R₁ represents O, NRi₆ with R₁₆ being H or an alkyl having from 1 to 4 carbon atoms, or N—(CH₂)ₚ—R₇, with R₇ representing OH, COOR₁₆, CO—NHR₁₆, NHR₁₆, O—CH₂R₁₆, CN, 0-CO—CH₂R₁₆, CO-heterocycloalkyl, a cycloalkyl, a heterocycloalkyl, an aryl or a heteroaryl, possibly substituted with one or more of the followings radicals: an alkyl, an aryl, a heteroaryl, a cycloalkyl, a heterocycloalkyl, F, Cl, NH₂, C≡_N, OH, CO—NH₂, 0-CH₃, CO—OH, CO—OCH₃ or 0-CO—CH₃, and p=0, 1, 2, 3, 4, 5 or 6 and with R₁₆ being H or an alkyl with 1, 2, 3 or 4 carbon atoms;
wherein R₂ represents H or an alkyl with less than 4 carbon atoms,
wherein R₃ represents S, O or NH in formula (a), (CH₂)_{q}—R₁₀ or NH—(CH₂)_{q}—R₁₀, in formula (a¹), wherein R₁₀ has the same meaning as R₉ and wherein q=O, 1, 2, 3 or 4;
wherein R₄ represents an alkyl, aryl, arylalkyl, heteroaryl, heteroarylalkyl, cycloalkyl, heterocycloalkyl, or CH═CH—R₁₁, wherein R₁₁ has the same meaning as R₄, and the molecular weight of R₄ is less than 500 Da,
and wherein,
if R₃ represents O, then R is N*—R₅, or 0-R₅, wherein R₅ has the same meaning as R₄ and N* represents NH or N(CH₃); if R₃ represents NH, S—(CH₂)_{q}—R₁₀ or NH—(CH₂)_{q}—Ri₀, then R is R₆, or N*—CO—R₆, wherein R₆ has the same meaning as R₄ and N* represents NH or N(CH₃); and if R₃ represents S, then *R is N*—(CH₂)ₙ—R₁₂, wherein R₁₂ has the same meaning as R₇, n=0, 1, 2, 3 or 4 and N* represents NH or N(CH₃), or
R is N*—CO—R₈, with -R₈ representing a linear or branched alkyl, with the exception of tert-butyl, or -R₈ represents —(CH₂)ₛ—C₄H₃O, with s=1, 2, 3 or 4, or R₈ represents a phenyl radical mono-substituted in the para position, with an alkyl, —F, —Cl, —NH₂, —C≡N, —OH₁—CO—NH₂, -0-CH₃, —CO—OH, —CO—OCH₃ or -0-CO—CH₃ and N* represents NH or N(CH₃), or
R is N*—C0-R₁₇, with -R₁₇ representing an alkyl, aryl, arylalkyl, heteroaryl, heteroarylalkyl, cycloalkyl, heterocycloalkyl, or CH═CH—R₁₈, wherein R₁₈ has the same meaning as R₁₇, and the molecular weight of R₁₇ is less than 500 Da, N* represents NH or N(CH₃) and R₁ is not O,
or base or acid addition salt of said compound, or a geometric isomer, an enantiomer or a diastereoisomer thereof.

2. A compound according to claim 1, wherein R₁ is an oxygen atom.

3. A compound according to claim 1, wherein R₂ is a methyl.

4. A compound according to claim 1, wherein at least one of cycles (I) and (II) is substituted with F or with (CH₂)ₘ—R₉, with m=1 or 2 and -[R₉] is

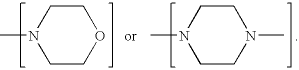

5. A compound according to claim 1, wherein in formula (a) R₁ is O, -R₂ is —CH₃, R₃ is O, Y is H, R is NH—R₅ and R₅ is a phenyl or R₅ is a benzyl or -R₅ is —C₆H₄—N(CH₃)₂.

6. A compound according to claim 1, wherein in formula (a) R₁ is O or NH, -R₂ is —CH₃, R₃
is NH, Y is H and -[R] is 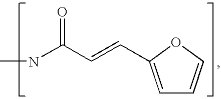
the furan moiety being optionally replaced by another heterocycloalkyl, e.g. thiophene, pyrrole, thiazole, pyrrazole or oxazole moiety, said moiety being optionally substituted with an alkyl having from 1 to 4 carbon atoms, NH₃, O, OH, CO—NH₂, 0-CH₃, CO—OH, CO—OCH₃ or 0-CO—CH₃.

7. A compound according to claim 1, wherein in formula (b) R₁ is O, -R₂ is —CH₃, Y is H and -[R₄] is

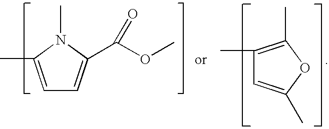

8. A compound according to claim 1, wherein in formula (a) R₁ is O or NH, -R₂ is —CH₃, R₃ is NH₁ Y is H and -R is —NH—CO—C₆H₅.

9. A compound according to claim 1, wherein in formula (a) R₁ is O, R₂ is H or CH₃, R₃ is S or O, Y is H, R is NH—(CH₂Jₙ-R₁₂, n=0 and f R₁₂] is

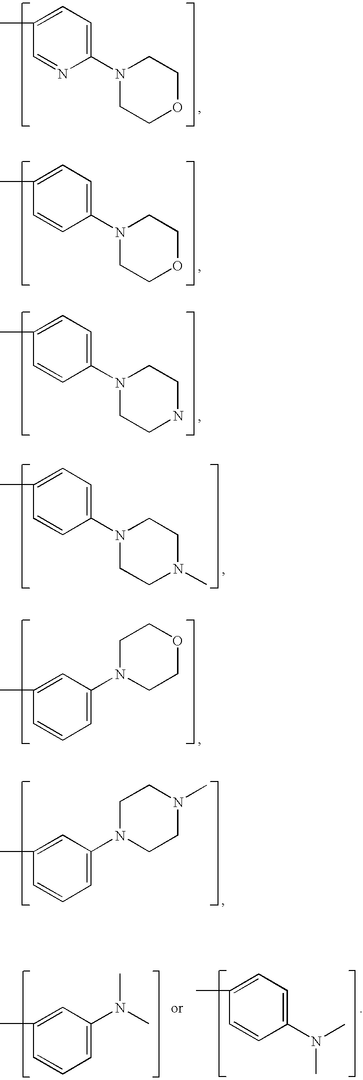

10. A compound according to claim 1, wherein in formula (a) R₁ is O, -R₂ is —CH₃, R₃ is S, Y is H, R is NH—(CH₂)ₙ—R₁₂, n=0 and R₁₂ is C₆H₅.

11. A compound according to claim 1, wherein in formula (a) R₁ is O, -R₂ is —CH₃, R₃ is S, Y is H, R is NH—CO—R₈ and -R₈ is —CH₂—CH₂—C₄H₃O or
R₈ ] is 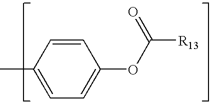
is wherein R₁₃ is an alkyl in C₁ to C₆.

12. A compound according to claim 1, wherein in formula (a) R₁ is O₁—R₂ is —CH₃, R₃ is S, R is NH—(CH₂)ₙ—R7, n=1 and -R₇ is —C₆H₅.

13. A compound according to claim 1, wherein in formula (a¹) R₁ is O, -R₂ is —CH₃, R₃ is S—CH₃ and {R] is 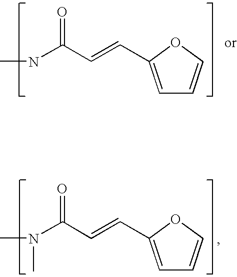
the furan moiety being optionally replaced by another heterocycloalkyl, e.g. thiophene, pyrrole, thiazole, pyrrazole or oxazole moiety, said moiety being optionally substituted with an alkyl having from 1 to 4 carbon atoms, NH₃, O, OH, CO—NH₂, 0-CH₃, CO—OH, CO—OCH₃ or 0-CO—CH₃.

14. A compound according to claim 1, wherein the compound is:
Compound 03: 1-[3-methoxy-4-(2-oxo-2/-/-chromen-3-yl)phenyl]-3-phenyl urea,
Compound 04: 1-[3-methoxy-4-(2-oxo-2H-chromen-3-yl)phenyl]-3-phenylthiourea,
Compound 05: 4-fluoro-/\/-({[3-methoxy-4-(2-oxo-2/-/-chromen-3-yl)phenyl]amino}carbonothioyl)benzamide,
Compound 06: 1-[4-(dimethylamino)phenyl]-3-[3-methoxy-4-(2-oxo-2/-/-chromen-3-yl)phenyl]urea,
Compound 07: /\/-(imino{[3-methoxy-4-(2-oxo-2/-/-chromen-3-yl)phenyl]amino}methyl)benzamide,
Compound 08: 1-[3-methoxy-4-(2-oxo-2H-chromen-3-yl)phenyl]-3-(6-morpholin-4-ylpyridin-3-yl)thiourea,
Compound 09: methyl 5-({[3-methoxy-4-(2-oxo-2H-chromen-3-yl)phenyl]amino}sulfonyl)-1-methyl-1/-/-pyrrole-2-carboxylate,
Compound 10: methyl /\/-[(2E)-3-(2-furyl)prop-2-enoyl]-/\/′-[3-methoxy-4-(2-oxo-2H-chromen-3-yl)phenyl]-/\/-methylimidothiocarbamate,
Compound 12: (2E)-3-(2-furyl)-/\/-(imino{[3-methoxy-4-(2-oxo-2/-/-chromen-3-yl)phenyl]amino}methyl)acrylamide,
Compound 13: /\/-[3-methoxy-4-(2-oxo-2H-chromen-3-yl)phenyl]-2,5-dimethylfuran-3-sulfonamide,
Compound 14: methyl W-[(2E)-3-(2-furyl)prop-2-enoyl]-W-[3-methoxy-4-(2-oxo-2H-chromen-3-yl)phenyl]imidothiocarbamate,
Compound 15: (2E)-3-(2-furyl)-/\/-({[3-methoxy-4-(2-oxo-1,2-dihydroquinolin-3-yl)phenyl]amino}carbonothioyl)acrylamide,
Compound 16: methyl /\/-(4-fluorobenzoyl)-/\/-[3-methoxy-4-(2-oxo-2/-/-chromen-3-yl)phenyl]-/\/-methylimidothiocarbamate,
Compound 17: (2E)-3-(2-furyl)-/\/-({[3-methoxy-4-(1-methyl-2-oxo-1,2-dihydroquinolin-3-yl)phenyl]amino}carbonothioyl)acrylamide,
Compound 18: 1-[3-(dimethylamino)phenyl]-3-[3-methoxy-4-(2-oxo-2H-chromen-3-yl)phenyl]urea,
Compound 19: N′-((E)-3-furan-2-yl-acryloyl)-N-[3-methoxy-4-(2-oxo-2H-chromen-3-yl)-phenyl]-N-methyl-guanidine,
Compound 20: 1-[3-methoxy-4-(2-oxo-2H-chromen-3-yl)-phenyl]-3-(4-morpholin-4-yl-phenyl)-thiourea,
Compound 21: 1-((E)-3-furan-2-yl-acryloyl)-3-{4-[1-(2-hydroxy-ethyl)-2-oxo-1,2-dihydro-quinolin-3-yl]-3-methoxy-phenyl}-thiourea,
Compound 22: N-Benzoyl-N′-[3-methoxy-4-(2-oxo-1,2-dihydro-quinolin-3-yl)-phenyl]-guanidine,
Compound 23: 2,5-dimethyl-furan-3-sulfonic acid acetyl-[3-methoxy-4-(2-oxo-2H-chromen-3-yl)-phenyl]-amide,
Compound 24: 1-((E)-3-furan-2-yl-acryloyl)-3-[3-methoxy-4-(2-oxo-2H-chromen-3-yl)-phenyl]-1,2-dimethyl-isothiourea, or
Compound 25: 1-(4-fluoro-benzoyl)-3-[3-methoxy-4-(2-oxo-2H-chromen-3-yl)-phenyl]-1,2-dimethyl-isothiourea.

15. Compound with antiproliferative properties having the general formula: 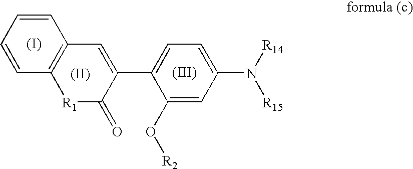
formula (a)—wherein the cycles (I), (III) and (III) may be optionally substituted on 1, 2, 3 or 4 of the available carbons with a group chosen amongst: Br, Cl, F, I₁OH, 0-CH₃, COOH, and (CH₂)ₘ—R₉, wherein R₉ represents H, CH₃, OH, 0-CH₃, COOH₁ NH₃, CO—NH₂, NH—CO— phenyl, an alkyl, an heteroaryl or an heterocycloalkyl and m=1, 2, 3 or 4;
wherein R₁ represents O, NR₁₆ with R₁₆ being H or an alkyl having from 1 to 4 carbon atoms, or N—(CH₂)ₚ—R₇, with R₇ representing OH, COOR₁₆, CO—NHR₁₆, NHR₁₆, O—CH₂R₁₆, CN, 0-CO—CH₂Ri₆, CO-heterocycloalkyl, a cycloalkyl, a heterocycloalkyl, an aryl or a heteroaryl, possibly substituted with one or more of the followings radicals: an alkyl, an aryl, a heteroaryl, a cycloalkyl, a heterocycloalkyl, F, Cl, NH₂, C≡N, OH, CO—NH₂, 0-CH₃, CO—OH, CO—OCH₃ or 0-CO—CH₃, and p=0, 1, 2, 3, 4, 5 or 6 and with R₁₆ being H or an alkyl with 1, 2, 3 or 4 carbon atoms;
wherein R₂ represents H or an alkyl with less than 4 carbon atoms,
wherein R₁₄ and R₁₅ are, independently, any type of substituent with a molecular weight less than 400 Dalton, provided that R₁₄ and Ri₅ are not simultaneously oxygen, or base or acid addition salt of said compound, or a geometric isomer, an enantiomer or a diastereoisomer thereof, for use in therapy.

16. A compound according to claim 15, wherein at least one of R₁₄, R₁₅, NR₁₄ and NR₁₅ contains an amide, a sulphonamide, a ketone, a furan, an urea, thiourea group, a benzyl, a phenyl, an acyl or a guanidine.

17. A compound according to claim 15, wherein at least one of cycles (I) and (II) is 2 and -[R₉] is

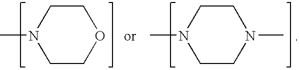

18. A compound according to claim 15, wherein R₁₄ and R₁₅ are different.

19. A compound according to claim 15, wherein at least one of R₁₄ and R₁₅ is H or CH₃.

20. A compound according to claim 15, wherein the compound is: Compound 01 (2£)-3-(2-furyl)-/\/-({[3-methoxy-4-(2-oxo-2H-chromen-3-yl)phenyl]amino}carbo-nothioyl)acrylamide,
Compound 02: /\/-({[3-methoxy-4-(2-oxo-2H-chromen-3-yl)phenyl]amino}carbonothioyl)benzamide.
Compound 03: 1-[3-methoxy-4-(2-oxo-2H-chromen-3-yl)phenyl]-3-phenylurea,
Compound 04: 1-[3-methoxy-4-(2-oxo-2H-chromen-3-yl)phenyl]-3-phenylthiourea,
Compound 05: 4-fluoro-/\/-({[3-methoxy-4-(2-oxo-2/-/-chromen-3-yl)phenyl]amino}carbonothioyl)benzamide,
Compound 06: 1-[4-(dimethylamino)phenyl]-3-[3-methoxy-4-(2-oxo-2H-chromen-3-yl)phenyl]urea,
Compound 07: W-(imino{[3-methoxy-4-(2-oxo-2H-chromen-3-yl)phenyl]amino}methyl)benzamide,
Compound 08: 1-[3-methoxy-4-(2-oxo-2H-chromen-3-yl)phenyl]-3-(6-morpholin-4-ylpyridin-3-yl)thiourea,
Compound 09 methyl 5-({[3-methoxy-4-(2-oxo-2H-chromen-3-yl)phenyl]amino}sulfonyl)-1-methyl-1H-pyrrole-2-carboxylate,
Compound 10: methyl /\/-[(2E)-3-(2-furyl)prop-2-enoyl]-/\/¹-[3-methoxy-4-(2-oxo-2H-chromen-3-yl)phenyl]-/\/-methylimidothiocarbamate,
Compound 12: (2E)-3-(2-furyl)-/\/-(iminoflS-methoxy-̂-̂-oxo-̂H-chromen-S-yOphenyllaminoJmethylJacrylamide,
Compound 13: /\/-[3-methoxy-4-(2-oxo-2H-chromen-3-yl)phenyl]-2,5-dimethylfuran-3-sulfonamide,
Compound 14: methyl A/-[(2E)-3-(2-furyl)prop-2-enoyl]-/\/-[3-methoxy-4-(2-oxo-2H-chromen-S-yOphenyφmidothiocarbamate,
Compound 15: (2£)-3-(2-furyl)-/\/-({[3-methoxy-4-(2-oxo-1,2-dihydroquinolin-3-yl)phenyl]amino}carbonothioyl)acrylamide,
Compound 16: methyl /\/-(4-fluorobenzoyl)-/\/′-[3-methoxy-4-(2-oxo-2/-/-chromen-3-yl)phenyl]-/\/-methylimidothiocarbamate,
Compound 17: (2£)-3-(2-furyl)-/\/-({[3-methoxy-4-(1-methyl-2-oxo-1,2-dihydroquinolin-3-yl)phenyl]amino}carbonothioyl)acrylamide,
Compound 18: 1-[3-(dimethylamino)phenyl]-3-[3-methoxy-4-(2-oxo-2H-chromen-3-yl)phenyl]urea,
Compound 19: N′-((E)-3-furan-2-yl-acryloyl)-N-[3-methoxy-4-(2-oxo-2H-chromen-3-yl)-phenyl]-N-methyl-guanidine,
Compound 20: 1-[3-methoxy-4-(2-oxo-2H-chromen-3-yl)-phenyl]-3-(4-morpholin-4-yl-phenyl)-thiourea,
Compound 21: 1-((E)-3-furan-2-yl-acryloyl)-3-{4-[1-(2-hydroxy-ethyl)-2-oxo-1,2-dihydro-quinolin-3-yl]-3-methoxy-phenyl}-thiourea,
Compound 22: N-Benzoyl-N′-[3-methoxy-4-(2-oxo-1,2-dihydro-quinolin-3-yl)-phenyl]-guanidine,
Compound 23: 2,5-dimethyl-furan-3-sulfonic acid acetyl-[3-methoxy-4-(2-oxo-2H-chromen-3-yl)-phenyl]-amide,
Compound 24: 1-((E)-3-furan-2-yl-acryloyl)-3-[3-methoxy-4-(2-oxo-2H-chromen-3-yl)-phenyl]-1,2-dimethyl-isothiourea, or
Compound 25: 1-(4-fluoro-benzoyl)-3-[3-methoxy-4-(2-oxo-2H-chromen-3-yl)-phenyl]-1,2-dimethyl-isothiourea.

21. A compound according to claim 1, having cytotoxic properties on tumoral cells.

22. A compound according to claim 1, having cytostatic properties on tumoral cells.

23. A compound according to claim 21, wherein said tumoral cells are lung, prostate, breast, colon, melanoma, lymphoma or myeloid tumor cells.

24. A compound according to claim 1 which is soluble in water at molarity above 50.10⁻⁶ mol·L⁻¹., preferably at molarity above 500.10^{″6} mol·L^{′1}.

25. A compound according to claim 1 which binds Bcl-xL protein with a Kd less than 1 μM, preferably less than 10O nM.

26. A composition comprising a compound according to claim 1 in association with at least one therapeutic agent, preferentially one other anti-tumor agent.

27. A composition according to claim 26, wherein said anti-tumor agent is taxol, etoposide, doxorubicin, cis-platin, retinoic acid, tamoxifen, vinblastin, vincristin, cyclophosphamide, topotecan, irinotecan, gemcitabine or rapamycin.

28. A product containing a compound according to claim 1 and a further anti-tumor agent as a combined preparation for simultaneous, separate or sequential use in therapy.

29. A pharmaceutical composition comprising a compound according to claim 1 and a pharmaceutically acceptable carrier.

30. A pharmaceutical composition according to claim 29, which is to be administered via intralesional, intraperitoneal, intramuscular or intravenous injection; via infusion; liposome-mediated delivery; topical, nasal, oral, anal, subcutaneous, vaginal, sublingual, uretheral, transdermal, intrathecal, ocular or otic delivery.

31. (canceled)

32. Method according to claim 36, wherein said compound is administered via intralesional, intraperitoneal, intramuscular or intravenous injection; via infusion; liposome-mediated delivery; topical, nasal, oral, anal, subcutaneous, vaginal, sublingual, uretheral, transdermal, intrathecal, ocular or otic delivery.

33. Use of a composition according to claim 26, for the manufacture of a medicament to be administered to a subject for treating cancer.

34. The method according to claim 36, wherein the compound is administered simultaneously, separately or sequentially with at least one other anti-tumor agent.

35. Process for inhibiting cell proliferation in vitro comprising contacting a cell in vitro, with a compound according to claim 1.

36. A method of treating cancer in a patient in need of such treatment, comprising administering to the patient an anti-cancer effective amount of a compound of claim 1.
